(19) United States
(12) Patent Application Publication
(10) Pub. No.: US 2004/0048319 A1
(43) Pub. Date: Mar. 11, 2004

(54) ALCAM and ALCAM modulators

(76) Inventors: Jennie P. Mather, Millbrae, CA (US); Ronghao Li, Millbrae, CA (US); Mary C. Tsao, Burlingame, CA (US); Deryk T. Loo, Belmont, CA (US)

Correspondence Address: MORRISON & FOERSTER LLP, 755 PAGE MILL RD, PALO ALTO, CA, 94304-1018, US

(21) Appl. No.: 10/428,983

(22) Filed: May 2, 2003

Related U.S. Application Data
(60) Provisional application No. 60/377,479, filed on May 3, 2002.

(51) Int. Cl. G01N 33/574; C07H 21/04; C07K 16/30; C12N 5/06

(52) U.S. Cl. 435/7.23; 435/69.1; 435/320.1; 435/344; 530/388.8; 536/23.53

(57) ABSTRACT

Provided herein is disclosure about the discovery that the known antigen, ALCAM, is present on a variety of human cancers other than metastasizing malignant melanoma. Also disclosed herein are methods of diagnosing and treating cancers that express ALCAM by using antibodies directed against ALCAM. This invention also discloses compositions and methods for the modulation of ALCAM-mediated neovascularization.

### CROSS-REFERENCE TO RELATED APPLICATIONS

This application claims the benefit of U.S. provisional application serial No. 60/377,479, filed May 3, 2002, which is incorporated in its entirety by reference.

### TECHNICAL FIELD

This invention is in the fields of biology and immunotherapy. More specifically, it concerns the discoveries that ALCAM, a known antigen, is associated with a variety of human cancers other than melanomas and that anti-ALCAM antibodies may be used to treat such cancers. It also concerns ALCAM-mediated neovascularzation.

### BACKGROUND OF THE INVENTION

Activated leukocyte cell adhesion molecule (ALCAM) is a type I transmembrane protein and member of the immunoglobulin superfamily. ALCAM, also referred to/known as CD166, KG-CAM, or neurolin, has a short cytoplasmic tail and an extracellular portion with five Ig domains, two N-terminal variable type domains followed by three constant type Ig domains (Ohneda et al., (2001) Blood 98(7):2134-2142). ALCAM has over 90% homology with the chicken adhesion molecule BEN/SC1/DM-GRASP and 30% identity and 50% similarity with the human melanoma cell adhesion molecule Mel-CAM/MUC18/CD146 (Leon et al., (2001) J Biol Chem 276(28):25783-25790).

ALCAM has been found to be expressed on subsets of activated leukocytes, fibroblasts, and epithelial and neural cells, where ALCAM expression was correlated with clustering of cells. ALCAM is thought to be involved in multiple processes in development, including hematopoiesis, thymus development, the immune response system, neurite extension, neural cell migration, and osteogenesis.

Immunohistochemical studies have shown that ALCAM is expressed in embryonic endothelium and dorsal aorta. Activation of ALCAM in embryonic endothelium results in hematopoiesis, endothelial cell development and the formation of endothelial tubes (Ohneda et al. (2001). Blood 98(7):2134-2142). The capability of embryonic cells to stimulate neovascularization, as well as hematopoiesis, is not present in adult cells that do not express ALCAM.

Neovascularization is the growth of new blood vessels in a tissue. Typically, new vasculature is composed primarily of endothelial cells (i.e., cells normally found in and composing blood vessels) whose growth was signaled by various angiogenesis factors. In tumors, particularly those cancers that are growing rapidly and aggressively as compared to surrounding normal tissue, the growth of new vasculature has been observed. Some have thought that this new vasculature was produced as a result of signaling agents produced by tumor cells, and that such agents would presumably act to stimulate the growth of blood vessels into the tumor. It has been believed that this new tumor vasculature was typically composed of endothelial cells normally associated with blood vessels. Research groups have attempted to block this tumor angiogenesis with various agents (e.g. endostatin and angiostatin) that inhibit the formation of normal vessels.

An alternative approach to the blocking of tumor angiogenesis involves the identification of agents that selectively block formation of neovasculature formation in or around tumors without affecting normal vessel formation in healthy tissue. One such approach is based on the findings of new vasculature produced from tumor cells, rather than from endothelial cells entering from outside the tumor, in a process termed “vascular mimicry” (see e.g., Hendrix et al (2000) Breast Cancer Res. 2:417-422).

ALCAM was first identified as a ligand of CD6. The association of ALCAM with CD6 is thought to be involved in T cell interactions with monocytes and other activated T cells, as well as epithelial cells, fibroblasts and specialized cells, such as pancreatic islet cells. ALCAM is also expressed on monocyte-lineage cells in the inflamed synovium of individuals with rheumatoid arthritis (Levesque et al., (1998) Arthiritis Rheum 41(12):2221-9). The localization of ALCAM-CD6 interactions suggests a role in the maintenance of chronic inflammatory responses, in conditions such as rheumatoid arthritis (U.S. Pat. No. 5,998,172). Although certain binding interactions involving ALCAM have been described and ALCAM-related events are beginning to be elucidated, the mechanisms by which ALCAM acts remain to be determined. Identification of the event(s) that allow for ALCAM interactions with signaling and other molecules will greatly enhance the understanding of ALCAM function and will provide for agents that can modulate ALCAM function. Such agents will be useful for the treatment and diagnosis of a spectrum of pathologies, including those related to the processes described above and in the following paragraphs. There is a need for greater understanding of events with allow for the interaction of ALCAM with its native ligands and resultant biological events, and for peptides and other structures that can modulate these events.

In addition to the heterotypic ALCAM-CD6 cell-cell interaction, ALCAM is capable of forming homotypic ALCAM-ALCAM interactions as well, as observed in human metastatic melanoma cell lines. It has been previously shown that metastasizing malignant melanoma cells express ALCAM, whereas ALCAM was not detected in non-metastasizing malignant melanoma cells. The expression of ALCAM correlated with the progression of melanoma from a non-invasive radial growth phase to a vertical growth phase capable of metastasis. Anti-ALCAM antibody has been used as a diagnostic marker of primary malignant melanoma tumor progression (Van kempen et al., (2000) American J Path 156(3):769-774).

In addition to diagnostics, antibodies may be useful as therapeutic agents. For example, immunotherapy, or the use of antibodies for therapeutic purposes, has been used in recent years to treat cancer. Passive immunotherapy involves the use of antibodies, usually monoclonal antibodies, in cancer treatments. See for example, Cancer: Principles and Practice of Oncology, 6^{th} Edition (2001) Chapt. 20 pp. 495-508. These antibodies can have inherent therapeutic biological activity both by direct inhibition of tumor cell growth or survival and by their ability to recruit the natural cell killing activity of the body's immune system. These agents can be administered alone or in conjunction with radiation or chemotherapeutic agents. The monoclonal antibodies sold under the trademarks Rituxan® and Herceptin®, approved for treatment of lymphoma and breast cancer, respectively, are two examples of such therapeutics. Alternatively, antibodies can be used to make antibody conjugates where the antibody is linked to a toxic agent and directs that agent to the tumor by specifically binding to the tumor. The antibody conjugate sold under the trademark Mylotarg® is an example of an approved antibody conjugate used for the treatment of leukemia. Monoclonal antibodies that bind to cancer cells and have potential uses for diagnosis and therapy have been disclosed in publications. See, for example, the following patent applications which disclose, inter alia, some molecular weights of target proteins: U.S. Pat. No. 6,054,561 (200 KD c-erbB-2 (Her2), and other unidentified antigens 40-200 KD in size) and U.S. Pat. No. 5,656,444 (50 KD and 55 KD, oncofetal protein). Example of antibodies in clinical trials and/or approved for treatment of solid tumors include: Herceptin® (antigen: 180 kD, HER2/neu), Panorex® (antigen: 40-50 kD, Ep-CAM), HMFG1 (antigen >200 kD, HMW Mucin), C225 (antigens: 150 kD and 170 kD, EGF receptor), Campath® (antigen: 21-28 kD, CD52), and Rituxan® (antigen: 35 kD, CD20). The antigen targets of Herceptin® (Her-2 receptor), which is used to treat breast cancer and C225 (EGF receptor) in clinical trials for the treatment of several cancers, are present at some detectable level on a large number of normal human adult tissues including, skin, colon, lung, ovary, liver, and pancreas. The margin of safety in using these therapeutics is possibly provided by difference in the level of expression or in access of or activity of the antibody at these sites. As of April 2002, publicly available antibodies which are known to bind specifically to ALCAM include clone 18 from Antigenix America Inc., New York; clone 3A6 from Ancell Corporation, Minnesota; clone j3-119 from Chromaprobe Inc., California; clone L50 from Caltag Laboratories Inc., California; catalog number AF656 from R&D Systems, Inc., Minnesota; and catalog number sc-8548 and sc-8549 from Santa Cruz Biotechnology, California, as listed in Linscott's Directory of Immunological and Biological Reagents (ISSN:0740-7394).

What is needed are novel targets on the surface of non-melanoma cancer cells that may be used to treat such cancers with antibodies and other agents which specifically recognize the non-melanoma cancer cell surface targets. There exists a further need, based on the discoveries disclosed herein, for novel antibodies and other agents which specifically recognize targets on the surface of cells that can modulate, either by reducing or enhancing, the newly discovered neovascularization activities of ALCAM.

### SUMMARY OF THE INVENTION

The invention disclosed herein concerns the discoveries that the known antigen, ALCAM, is present on a variety of both primary and metastatic human cancers, other than metastatic malignant melanoma, and that anti-ALCAM antibodies may be used to treat such cancers. In addition, ALCAM agonists and antagonists are provided to modulate neovascularization.

In one aspect, the invention is a composition comprising an anti-ALCAM antibody bound to ALCAM present on a cancer cell. In preferred embodiments, the cancer cell is selected from the group consisting of ovarian, lung, prostate, pancreatic, colon, and breast cancer cells. In some embodiments, the cancer cell is isolated. In some embodiments, the cancer cell is in a biological sample. Generally, the biological sample is from an individual, such as a human. In some embodiments, the anti-ALCAM antibody is linked to a therapeutic agent or a detectable label.

In some embodiments, the invention is an anti-ALCAM antibody mKID2 that is produced by a host cell (ATCC No. PTA-4478) or progeny thereof.

In another aspect, the invention is an anti-ALCAM antibody or a polypeptide (which may or may not be an antibody) that competitively inhibits preferential binding of an anti-ALCAM antibody to ALCAM. In some embodiments, the invention is an antibody or a polypeptide (which may or may not be an antibody) that binds preferentially to the same or different epitopes on ALCAM as other anti-ALCAM antibodies.

In another aspect, the invention is an antibody comprising a fragment or a region of an anti-ALCAM antibody. In one embodiment, the fragment is a light chain of the antibody. In another embodiment, the fragment is a heavy chain of the antibody. In yet another embodiment, the fragment contains one or more variable regions from a light chain and/or a heavy chain of the antibody. In yet another embodiment, the fragment contains one or more complementarity determining regions (CDRs) from a light chain and/or a heavy chain of the antibody.

In another aspect, the invention provides polypeptides (which may or may not be antibodies) comprising any of the following: a) one or more CDRs (or fragments thereof) from the light or heavy chain; b) three CDRs from the light chain; c) three CDRs from the heavy chain; d) three CDRs from the light chain and three CDRs from the heavy chain; e) the light chain variable region; f) the heavy chain variable region of the anti-ALCAM antibody.

In another aspect, the invention is a humanized antibody. In some embodiments, the humanized antibody comprises one or more CDRs of a non-human anti-ALCAM antibody. In some embodiments, the humanized antibody binds to the same or different epitope(s) as other anti-ALCAM antibodies. Generally, a humanized antibody of the invention comprises one or more (one, two, three, four, five, six, or fragments thereof) CDRs which are the same and/or derived from the CDR(s) of the original non-human anti-ALCAM antibody. In some embodiments, the human antibody binds to the same or different epitope(s) as other anti-ALCAM antibodies. In anther aspect, the invention is a chimeric antibody comprising variable regions derived from variable regions of a heavy chain and a light chain of a non-human anti-ALCAM antibody and constant regions derived from constant regions of a heavy chain and a light chain of a human antibody.

In yet another aspect, the invention is a host cell (ATCC No. PTA-4478) which produces monoclonal antibody mKID2.

In another aspect, the invention is an isolated polynucleotide that encodes for antibody mKID2 that is produced by a host cell with a deposit number of ATCC No. PTA-4478 or progeny thereof. In another aspect, the invention provides polynucleotides encoding any of the antibodies (including antibody fragments) as well as any other polypeptides described herein.

In another aspect, the invention is a pharmaceutical composition comprising any of the polypeptides (including any of the antibodies described herein) or polynucleotides described herein, such as pharmaceutical compositions comprising an anti-ALCAM antibody linked to a chemotherapeutic agent, an antibody comprising a fragment of an anti-ALCAM antibody, a humanized antibody of a non-human anti-ALCAM antibody, a chimeric antibody comprising variable regions derived from variable regions of a non-human anti-ALCAM antibody and constant regions derived from constant regions of a human antibody, or a human antibody with one or more properties of a non-human anti-ALCAM antibody, or any of the anti-ALCAM antibody described herein linked to a chemotherapeutic agent (such as a radioactive moiety), and a pharmaceutically acceptable excipient.

In another aspect, the invention is a method of generating antibody mKID2 comprising culturing a host cell (ATCC No. PTA-4478) or progeny thereof under conditions that allow production of antibody mKID2, and purifying the antibody mKID2.

In another aspect, the invention provides methods of generating any of the antibodies (or polypeptides) described herein by expressing one or more polynucleotides encoding the antibody (which may be separately expressed as a single light or heavy chain, or both a light and a heavy chain are expressed from one vector) in a suitable cell, generally followed by recovering and/or isolating the antibody or polypeptides of interest.

In another aspect, the invention is a method for diagnosing whether an individual has cancer, comprising determining whether there is expression of ALCAM on selected cells from the individual, wherein the expression of ALCAM on said cells is indicative of said cancer. In some embodiments, the expression of ALCAM is determined using an anti-ALCAM antibody. In some embodiments, the method involves detecting the level of ALCAM expression from cells. The term “detection” as used herein includes qualitative and/or quantitative detection (measuring levels) with or without reference to a control.

In another aspect, the invention is a method for diagnosing whether an individual has ovarian cancer, comprising determining whether there is expression of ALCAM on ovarian cells from the individual, wherein the expression of ALCAM on said cells is indicative of said cancer. In some embodiments, the expression of ALCAM is determined using an anti-ALCAM antibody. In some embodiments, the anti-ALCAM antibody is mKID2. In some embodiments, the method involves detecting the level of ALCAM expression from cells. The term “detection” as used herein includes qualitative and/or quantitative detection (measuring levels) with or without reference to a control.

In another aspect, the invention is a method for diagnosing whether an individual has lung cancer, comprising determining whether there is expression of ALCAM on lung cells from the individual, wherein the expression of ALCAM on said cells is indicative of said cancer. In some embodiments, the expression of ALCAM is determined using an anti-ALCAM antibody. In some embodiments, the anti-ALCAM antibody is mKID2. In some embodiments, the method involves detecting the level of ALCAM expression from cells.

In another aspect, the invention is a method for diagnosing whether an individual has prostate cancer, comprising determining whether there is expression of ALCAM on prostate cells from the individual, wherein the expression of ALCAM on said cells is indicative of said cancer. In some embodiments, the expression of ALCAM is determined using an anti-ALCAM antibody. In some embodiments, the anti-ALCAM antibody is mKID2. In some embodiments, the method involves detecting the level of ALCAM expression from cells.

In another aspect, the invention is a method for diagnosing whether an individual has pancreatic cancer, comprising determining whether there is expression of ALCAM on pancreatic cells from the individual, wherein the expression of ALCAM on said cells is indicative of said cancer. In some embodiments, the expression of ALCAM is determined using an anti-ALCAM antibody. In some embodiments, the anti-ALCAM antibody is mKID2. In some embodiments, the method involves detecting the level of ALCAM expression from cells.

In another aspect, the invention is a method for diagnosing whether an individual has colon cancer, comprising determining whether there is expression of ALCAM on colon cells from the individual, wherein the expression of ALCAM on said cells is indicative of said cancer. In some embodiments, the expression of ALCAM is determined using an anti-ALCAM antibody. In some embodiments, the anti-ALCAM antibody is mKID2. In some embodiments, the method involves detecting the level of ALCAM expression from cells.

In another aspect, the invention is a method for diagnosing whether an individual has breast cancer, comprising determining whether there is expression of ALCAM on breast cells from the individual, wherein the expression of ALCAM on said cells is indicative of said cancer. In some embodiments, the expression of ALCAM is determined using an anti-ALCAM antibody. In some embodiments, the anti-ALCAM antibody is mKID2. In some embodiments, the method involves detecting the level of ALCAM expression from cells.

In another aspect, the invention is a method for aiding diagnosis of cancer (such as but not limited to ovarian, lung, prostate, pancreatic, colon, or breast cancer) in an individual comprising determining the expression of ALCAM in a biological sample from the individual. In some embodiments, the expression of ALCAM is determined using an anti-ALCAM antibody. In some embodiments, the anti-ALCAM antibody is mKID2. In some embodiments, the method is detecting the level of ALCAM expression from cells.

In yet another aspect, the invention is a method for delivering a chemotherapeutic agent to cancer cells in an individual comprising administering an effective amount of a composition comprising an anti-ALCAM antibody associated with (including linked to) a chemotherapeutic agent to the individual. In some embodiments, the cancer cells are (but are not limited to) ovarian, prostate, pancreatic, lung, colon, or breast cancer cells. In some embodiments, the anti-ALCAM antibody is a humanized antibody derived from mKID2 (generally, but not necessarily, comprising one or more partial or intact CDRs of the antibody mKID2). In some embodiments, the anti-ALCAM antibody is a human antibody with one or more properties of the antibody mKID2. In some embodiments, the chemotherapeutic agent (such as a toxin or a radioactive molecule) is delivered into the ovarian cancer cells (is internalized). In some embodiments, the agent is saporin.

In another aspect, the invention is a method of treating cancer in an individual comprising administering an effective amount of a composition comprising an anti-ALCAM antibody associated with (including linked to) a chemotherapeutic agent to the individual. In some embodiments, the cancer is selected from the group consisting of, but not limited to, ovarian, prostate, pancreatic, lung, colon, or breast cancer. In some embodiments, the anti-ALCAM antibody is a humanized antibody derived from mKID2 (generally, but not necessarily, comprising one or more CDRs of the antibody mKID2). In some embodiments, the anti-ALCAM antibody is a human antibody with one or more properties of the antibody mKID2. In some embodiments, the cancer treated is ovarian cancer and the chemotherapeutic agent (such as a toxin or a radioactive molecule) is delivered into the ovarian cancer cells (is internalized). In some embodiments, the agent is saporin.

In another aspect, the invention is a method of inhibiting growth and/or proliferation of cancer cells in vitro or in an individual comprising administering an effective amount of a composition comprising an anti-ALCAM antibody associated with (including linked to) a chemotherapeutic agent to the cell culture or sample, to the individual. In some embodiments, the cancer cells are (but are not limited to) ovarian, prostate, pancreatic, lung, colon, or breast cancer cells. In some embodiments, the anti-ALCAM antibody is a humanized antibody derived from mKID2 (generally, but not necessarily, comprising one or more partial or intact CDRs of the antibody mKID2). In some embodiments, the anti-ALCAM antibody is a human antibody with one or more properties of the antibody mKID2. In some embodiments, the cancer cells are ovarian cancer cells and the chemotherapeutic agent (such as a toxin or a radioactive molecule) is delivered into the ovarian cancer cells (is internalized). In some embodiments, the agent is saporin.

In another aspect, the invention is a method of delaying development of metastasis in an individual with cancer comprising administering an effective amount of a composition comprising an anti-ALCAM antibody associated with (including linked to) a chemotherapeutic agent to the individual. In some embodiments, the cancer is ovarian, prostate, pancreatic, lung, colon, or breast cancer. In some embodiments, the anti-ALCAM antibody is a humanized antibody derived from mKID2 (generally, but not necessarily, comprising one or more CDRs of the antibody mKID2). In some embodiments, the anti-ALCAM antibody is a human antibody with one or more properties of the antibody mKID2. In some embodiments, the cancer is ovarian cancer and the chemotherapeutic agent (such as a toxin or a radioactive molecule) is delivered into the ovarian cancer cells (is internalized). In some embodiments, the agent is saporin.

The present invention further provides methods for modulating, either by enhancing or reducing, the association of ALCAM with a cytoplasmic signaling partner. The association of ALCAM with a cytoplasmic signaling partner can be impacted by contacting an ALCAM molecule presenting on a cell surface, with an agent that modulates the binding of the signaling partner to ALCAM. Agents which block or reduce ALCAM association with its binding and/or signaling partners can be used to modulate biological and pathological processes which are involved in ALCAM-mediated neovascularization. Pathological processes involving this action include tumor-associated vascular growth.

Agents can be tested for their ability to block, reduce, enhance or otherwise modulate the association of ALCAM with a binding partner, such as an anti-ALCAM antibody or CD6. Specifically, an agent can be tested for the ability to modulate such an interaction by incubating a peptide comprising the ALCAM interaction site (typically in its native conformation as it exists on intact living cells) with a binding partner and a test agent, and determining whether the test agent reduces or enhances the binding of the binding partner to the ALCAM peptide. Agonists, antagonists, and other modulators are expressly contemplated.

In yet another aspect, the invention is a method to promote blood vessel generation in vitro or in an individual comprising administering an effective amount of a composition comprising an anti-ALCAM agent to the individual.

In yet another aspect, the invention is a method to promote blood vessel generation in vitro or in an individual comprising administering an effective amount of a composition comprising an anti-ALCAM antibody to the individual.

In yet another aspect, the invention is a method to inhibit blood vessel generation in vitro or in an individual comprising administering an effective amount of a composition comprising an antagonistic anti-ALCAM agent to the individual.

In yet another aspect, the invention is a method to inhibit blood vessel generation in vitro or in an individual comprising administering an effective amount of a composition comprising an antagonistic anti-ALCAM antibody to the individual.

In yet another aspect, the invention is a composition that promotes neovascularization comprising an agent that binds to ALCAM on non-endothelial cells.

In yet another aspect, the invention is a composition that inhibits neovascularization comprising an antagonist agent that binds to ALCAM on non-endothelial cells.

In yet another aspect, the invention is a composition that promotes neovascularization comprising an antibody that binds to ALCAM on non-endothelial cells.

In yet another aspect, the invention is a composition that inhibits neovascularization comprising an antagonistic antibody that binds to ALCAM on non-endothelial cells.

In yet another aspect, the invention is a method to inhibit blood vessel generation in vitro or in an individual comprising administering to an individual an effective amount of a composition comprising an antibody against a vascularization-promoting molecule.

In yet another aspect, the invention is a method to promote blood vessel generation in vitro or in an individual comprising administering an effective amount of a composition comprising at least one vascularization-promoting molecule to the individual.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows histogram plots of anti-ALCAM monoclonal antibody 2D4 binding to nine different pancreatic cancer cell lines. The cells in each cell line were sorted using a fluorescence activated cell sorter. In each histogram, the gray, filled-in curve represents the non-specific binding of anti-human IgG Fc to each respective cell line without primary antibody. The black line curve is a histogram of anti-ALCAM binding to each pancreatic cancer cell line.

FIG. 2 is a photograph that shows vascularization of nude mice grafted with Rav 9926 cells, a proprietary human pancreatic tumor cell line, under the kidney capsule. FIGS. 2A and 2B are photographs of control mouse kidney capsule injected with an excipient. FIGS. 2C and 2D are photographs of mouse kidney capsule injected with anti-ALCAM antibody clone 2D4.

FIG. 3 is a graph that shows the effect of mKID2 and MAb-ZAP (an anti-IgG conjugate to saporin) on the growth of human ovarian cancer cells SKOV3.

FIG. 4 is a graph that shows the effect of mKID2 (at concentrations of 1 microg/ml, 10 microg/ml, and 20 microg/ml) and MAb-ZAP on the growth of human ovarian cancer cells SKOV3.

### DETAILED DESCRIPTION OF THE INVENTION

The invention disclosed herein concerns the discoveries that the antigen ALCAM is present on a variety of human cancers, including but not limited to ovarian, prostate, pancreatic, lung, colon, and breast cancer. The invention provides antibodies and polypeptides that bind to ALCAM and methods of making and using these antibodies and polypeptides to diagnose such cancer and treat such cancer. Anti-ALCAM antibodies have been found to suppress growth of cancer cells in vitro by binding to the ALCAM present on the cell surface and leading to internalization of a therapeutic agent into the cancer cells.

I. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

II. Definitions

An “antibody” is an immunoglobulin molecule capable of specific binding to a target, such as a polypeptide, through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (ScFv), naturally occurring variants, fusion proteins comprising an antibody portion with an antigen recognition site of the required specificity, chimeric antibodies, such as humanized antibodies, and any other modified configuration of the immunoglobulin molecule that comprises an antigen recognition site of the required specificity.

A “monoclonal antibody” refers to a substantially homogeneous antibody population that may include naturally occurring variants, wherein the monoclonal antibody is comprised of amino acids (naturally-occurring and nonnaturally-occurring) that are involved in the selective binding of an antigen. Monoclonal antibodies are highly specific, being directed against a single antigenic site. The term is not intended to be limited as regards the source of the antibody or the manner in which it is made (e.g., by hybridoma, phage selection, recombinant expression, transgenic animals, etc.). The term includes whole immunoglobulins as well as the fragments, etc. described above under the definition of “antibody”.

A “humanized antibody” refers to a chimeric molecule, generally prepared using recombinant techniques, which contain nonhuman variable regions and human constant regions. This eliminates the constant region as an immunogen in human individuals, but the possibility of an immune response to the foreign variable region remains (LoBuglio, A. F. et al., (1989) Proc Natl Acad Sci USA 86:4220-4224). Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the antigens in question and determine binding capability, flanked by four framework regions (FRs) which are relatively conserved in a given species and which putatively provide a scaffolding for the CDRs. When nonhuman antibodies are prepared with respect to a particular antigen, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856. Riechmann, L., et al., (1988) Nature 332:323-327; Verhoeyen, M., et al., (1988) Science 239:1534-1536; Kettleborough, C. A., et al., (1991) Protein Engineering 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

An epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an ALCAM epitope is an antibody that binds this ALCAM epitope with greater affinity, avidity, more readily, and/or with greater duration than it binds to other ALCAM epitopes or non-ALCAM epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the terms “mKID2”, “antibody mKID2” and “monoclonal antibody mKID2” are used interchangeably to refer to immunoglobulin produced by a host cell with a deposit number of ATCC No. PTA-4478 or progeny thereof.

Different biological functions are associated with anti-ALCAM antibodies, including, but not limited to, ability to bind to ALCAM (including ALCAM on cancer cells, including but not limited to ovarian, prostate, pancreatic, lung, colon, or breast cancer cells); ability to bind to a portion of ALCAM that is exposed on the surface of a living cell in vitro or in vivo; ability to deliver a chemotherapeutic agent to cancerous cells (such as ovarian, prostate, pancreatic, lung, colon, or breast cancer cells) expressing ALCAM; ability to deliver a therapeutic agent or detectable marker into cancer cells (such as ovarian cancer cells) expressing ALCAM. As discussed herein, polypeptides (including antibodies) of the invention may have any one or more of these characteristics.

An “anti-ALCAM equivalent antibody” or “anti-ALCAM equivalent polypeptide” refers to an antibody or a polypeptide having one or more biological functions associated with an anti-ALCAM antibody, such as, for example binding specificity.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

An “effective amount” of a pharmaceutical composition, in one embodiment, is an amount sufficient to effect beneficial or desired results including, without limitation, clinical results such as shrinking the size of a tumor, retardation of cancerous cell growth, delaying the development of metastasis, decreasing symptoms resulting from the disease, increasing the quality of life of those suffering from the disease, decreasing the dose of other medications required to treat the disease, enhancing effect of another medication such as via targeting and/or internalization, delaying the progression of the disease, and/or prolonging survival of individuals with severe disease associated with cancer. In another embodiment, an effective amount is an amount sufficient to modulate (promote or inhibit) neovascularization in the case of treatment with an ALCAM agonist or antagonistic agent. An effective amount can be administered in one or more administrations. For purposes of this invention, an effective amount of a pharmaceutical composition is an amount sufficient to reduce the proliferation of (or destroy) cancerous cells and to reduce and/or delay the development of metastasis of cancerous cells, either directly or indirectly. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic agents, and a single agent may be considered to be given in an effective amount if, in conjunction with one or more other agents, a desirable result may be or is achieved. While individual needs vary, determination of optimal ranges of effective amounts of each component is within the skill of the art. Typical dosages comprise 0.1 to 100 mg/kg/body weight. The preferred dosages comprise 1 to 100 mg/kg/body weight. The most preferred dosages comprise 10 to 100 mg/kg/body weight.

As used herein, “treatment” or “treating” is an approach for obtaining beneficial or desired results including and preferably clinical results. For purposes of this invention, beneficial or desired clinical results include, but are not limited to, one or more of the following: reducing the proliferation of (or destroying) cancerous or other diseased cells, reducing metastasis of cancerous cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of individuals with cancer.

As used herein, “delaying development of metastasis” means to defer, hinder, slow, retard, stabilize, and/or postpone development of metastasis. This delay can be of varying lengths of time, depending on the history of the cancer and/or individual being treated. As is evident to one skilled in the art, a sufficient or significant delay can, in effect, encompass prevention, in that the individual does not develop the metastasis.

A “biological sample” encompasses a variety of sample types obtained from an individual and can be used in a diagnostic or monitoring assay. The definition includes blood and other liquid samples of biological origin, solid tissue samples such as a biopsy specimen or tissue cultures or cells derived therefrom, and the progeny thereof, for example, cells obtained from a tissue sample collected from an individual suspected of having cancer, in preferred embodiments from ovary, lung, prostate, pancreas, colon, and breast tissue. The term “biological sample” encompasses a clinical sample, and also includes cells in culture, cell supernatants, cell lysates, serum, plasma, biological fluid, and tissue samples.

As used herein, a nucleic acid molecule or agent, antibody, composition or cell, etc., is said to be “isolated” when that nucleic acid molecule, agent, antibody, composition, or cell, etc. is substantially separated from contaminant nucleic acid molecules, antibodies, agents, compositions, or cells, etc. from its original source.

An “individual” is a vertebrate, preferably a mammal, more preferably a human. Mammals include, but are not limited to, farm animals, sport animals, pets (such as cats, dogs, horses), primates, mice and rats.

As used herein, “agent” refers to a biological, pharmaceutical, or chemical compound. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. hi addition, various natural sources can provide compounds for screening, such as plant or animal extracts, and the like. A skilled artisan can readily recognize that there is no limit as to the structural nature of the agents of the present invention.

Agents that are employed in the methods of this invention can be randomly selected or rationally selected or designed. As used herein, an agent is said to be randomly selected when the agent is chosen randomly without considering the specific sequences involved in the association of ALCAM with its native binding partners or known antibodies. An example of randomly selected agents is the use of a chemical library or a peptide combinatorial library.

As used herein, an agent is said to be rationally selected or designed when the agent is chosen on a nonrandom basis that takes into account the sequence of the target site and/or its conformation in connection with the agent's action. With respect to anti-ALCAM agents, it is currently believed that there are at least three epitopes on ALCAM against which antibodies can be raised and therefore at least three sites of action for agents that block ALCAM/anti-ALCAM interaction. This invention also encompasses agents which act at the sites of interaction between ALCAM and its native binding partner, commonly known as CD6 (other native ligands and their active ALCAM-interactive sites are also encompassed within the scope of this invention, whether currently known or later identified). Agents can be rationally selected or rationally designed by utilizing the peptide sequences that make up the contact sites of the receptor/ligand and/or ALCAM/anti-ALCAM antibody pair. For example, a rationally selected peptide agent can be a peptide whose amino acid sequence is identical to an epitope appearing on ALCAM as it is exposed on the surface of a living cell in its native environment. Such an agent will reduce or block the association of the ALCAM antibody with ALCAM, or the association of ALCAM with CD6, as desired, by binding to the anti-ALCAM antibody or to CD6.

As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE)) to the antibody, as well as indirect labeling of the probe or antibody by reactivity with a detectable substance.

As used herein, the term “association”, with regard to the antibody, includes covalent and non-covalent attachment or binding to an agent (e.g., chemotherapeutic agent). The antibody can be associated with an agent (e.g., chemotherapeutic agent) by direct binding or indirect binding via attachment to a common platform, such that the antibody directs the localization of the agent to the cancerous cell to which the antibody binds and wherein the antibody and agent do not substantially dissociate under physiological conditions such that the agent is not targeted to the same cancerous cell to which the antibody binds or such that the agent's potency is not decreased.

III. Composition of Anti-ALCAM Antibodies

Antibodies known to be specific for activated leukocyte cell adhesion molecule (ALCAM) exist in the art, and include both polyclonal and monoclonal antibodies. As of April 2002, publicly available anti-ALCAM antibodies are available from R&D Systems, Inc.; clone 18 from Antigenix America Inc., New York; clone 3A6 from Ancell Corporation, Minnesota; clone j3-119 from Chromaprobe Inc., California; clone L50 from Caltag Laboratories Inc., California; catalog number AF656 from R&D Systems, Inc., Minnesota, as listed in Linscott's Directory of Immunological and Biological Reagents (ISSN:0740-7394) and polyclonal antibodies from Santa Cruz Biotechnology. These antibodies may be purchased, or alternatively, the antigen ALCAM may be purchased or otherwise obtained by conventional methods and used as an immunogen to generate additional antibodies against ALCAM. The techniques of using cells expressing ALCAM as an immunogen are described in further detail below.

This invention also encompasses compositions, including pharmaceutical compositions, comprising anti-ALCAM antibodies, polypeptides derived from anti-ALCAM antibodies, polynucleotides comprising sequence encoding anti-ALCAM antibodies, and other agents as described herein. Antibody mKID2 is an anti-ALCAM antibody suitable for use in the practice of this invention. It is produced from the hybridoma which has been deposited in the American Type Culture Collection (ATCC) 10801 University Blvd., Manassas Va. 20110-2209 on Jun. 21, 2002 with a Patent Deposit Designation of PTA-4478. As used herein, compositions further comprises one or more antibodies, polypeptides and/or proteins that bind to ALCAM, and/or one or more polynucleotides comprising sequences encoding one or more antibodies, polypeptides and proteins that bind to ALCAM. As will be discussed further below, compositions encompassed by this invention also include agents that enhance or reduce ALCAM-mediated neovascularization. Without being limited to any specific mechanism of action, these neovascularization modulators may act directly on ALCAM or act on its native or induced binding partners. CD6 is an example of a known binding partner that is suitable for modulation with agents that enhance or reduce ALCAM-mediated neovascularization, according to the teachings of this invention. Anti-CD6 antibodies that have the desired characteristics described herein may be utilized in the practice of this invention.

In addition to the pharmacologically active agent, the compositions of the present invention may contain suitable pharmaceutically acceptable carriers comprising excipients and auxiliaries which are well known in the art and which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulation may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

The antibodies, agents, polypeptides and proteins of this invention are further identified and characterized by any (one or more) of the following criteria: (a) ability to bind to ALCAM (including ALCAM on cancer cells, including but not limited to ovarian, prostate, pancreatic, lung, colon, or breast cancer cells); (b) ability to competitively inhibits preferential binding of a known anti-ALCAM antibody to ALCAM, including the ability to preferentially bind to the same ALCAM epitope to which the original antibody preferentially binds; (c) ability to bind to a portion of ALCAM that is exposed on the surface of a living cell in vitro or in vivo; (d) ability to bind to a portion of ALCAM that is exposed on the surface of living cancer cells, such as but not limited to ovarian, prostate, pancreatic, lung, colon, or breast cancer cells; (e) ability to deliver a chemotherapeutic agent or detectable marker to cancerous cells (such as but not limited to ovarian, prostate, pancreatic, lung, colon, or breast cancer cells) expressing ALCAM; (f) ability to deliver a therapeutic agent into cancerous cells (such as but not limited to ovarian cancer cells) expressing ALCAM.

In some embodiments, the antibody of the invention is an antibody mKID2 that is produced by a host cell with a deposit number of ATCC No. PTA-4478 or progeny thereof. The present invention also encompasses various formulations of mKID2 and equivalent antibodies or polypeptide fragments (e.g., Fab, Fab′, F(ab′)₂, Fv, Fc, etc.), chimeric antibodies, single chain (ScFv), mutants thereof, fusion proteins comprising an antibody portion, humanized antibodies, and any other modified configuration of mKID2 that comprises an antigen (ALCAM), recognition site of the required specificity. The invention also provides human antibodies displaying one or more of the biological characteristics of mKID2. The equivalent antibodies of mKID2 (including humanized antibodies and human antibodies), polypeptide fragments of mKID2, and polypeptides comprising any of these fragments are identified and characterized by any (one or more) of the five criteria described above.

In some embodiments, the antibodies, polypeptides and proteins of the invention that bind to ALCAM are antibodies, polypeptides and proteins that competitively inhibit preferential binding of mKID2 to ALCAM. In some embodiments, the antibodies, the polypeptides and the proteins preferentially bind to the same epitope on ALCAM as the antibody mKID2 preferentially binds.

Accordingly, the invention provides any of the following (or compositions, including pharmaceutical compositions, comprising any of the following): (a) antibody mKID2 produced by the host cell with a deposit number of ATCC No. PTA-4478 or its progeny; (b) a humanized form of antibody mKID2; (c) an antibody comprising one or more of the light chain and/or heavy chain variable regions of antibody mKID2; (d) a chimeric antibody comprising variable regions homologous or derived from variable regions of a heavy chain and a light chain of antibody mKID2, and constant regions homologous or derived from constant regions of a heavy chain and a light chain of a human antibody; (e) an antibody comprising one or more of the light chain and/or heavy chain CDRs (at least one, two, three, four, five, or six) of mKID2; (f) an antibody comprising a heavy and/or a light chain of mKID2; (g) a human antibody that is equivalent to mKID2. A humanized form of the antibody may or may not have CDRs identical to mKID2, or antibody produced by the host cell with a deposit number of ATCC No. PTA-4478. Determination of CDR regions is well within the skill of the art. In some embodiments, the invention provides an antibody which comprises at least one CDR that is substantially homologous to at least one CDR, at least two, at least three, at least four, at least 5 CDRs of mKID2 (or, in some embodiments substantially homologous to all 6 CDRs of mKID2, or derived from mKID2), or antibody produced by the host cell with a deposit number of ATCC No. PTA-4478. Other embodiments include antibodies that have at least two, three, four, five, or six CDR(s) that are substantially homologous to at least two, three, four, five or six CDRs of mKID2 or derived from mKID2, or antibody produced by the host cell with a deposit number of ATCC No. PTA-4478. It is understood that, for purposes of this invention, binding specificity and/or overall activity (which may be in terms of delivering a chemotherapeutic agent to or into cancerous cells to reduce the growth and/or proliferation of cancer cells, to induce apoptotic cell death in the cancer cell, to delay the development of metastasis, and/or treating palliatively) is generally retained, although the extent of activity may vary compared to mKID2 (may be greater or lesser). The invention also provides methods of making any of these antibodies. Methods of making antibodies are known in the art and are described herein.

The invention also provides polypeptides comprising an amino acid sequence of the antibodies of the invention, such as mKID2. In some embodiments, the polypeptide comprises one or more of the light chain and/or heavy chain variable regions of the antibody. In some embodiments, the polypeptide comprises one or more of the light chain and/or heavy chain CDRs of the antibody. In some embodiments, the polypeptide comprises three CDRs of the light chain and/or heavy chain of the antibody. In some embodiments, the polypeptide comprises an amino acid sequence of the antibody that has any of the following: at least 5 contiguous amino acids of a sequence of the original antibody, at least 8 contiguous amino acids, at least about 10 contiguous amino acids, at least about 15 contiguous amino acids, at least about 20 contiguous amino acids, at least about 25 contiguous amino acids, at least about 30 contiguous amino acids, wherein at least 3 of the amino acids are from a variable region of the antibody. In one embodiment, the variable region is from a light chain of the original antibody. In another embodiment, the variable region is from a heavy chain of the antibody. In another embodiment, the 5 (or more) contiguous amino acids are from a complementarity determining region (CDR) of the antibody.

IV. Methods of Producing Anti-ALCAM Antibodies

Methods of making antibodies against ALCAM are known in the art. Typically, monoclonal antibodies are developed in non-human species, such as mice. The antibodies are produced by immunizing mice with an immunogenic amount of cells, cell extracts, or protein preparations that contains human ALCAM (e.g. human thymic epithelial cells). Cells used for immunogen may be cultured for a period of time (at least 24 hours) prior to their use as an immunogen. Cells may be used as immunogens by themselves or in combination with a non-denaturing adjuvant, such as Ribi. In general, cells should be kept intact and preferably viable when used as immunogens. Intact cells may allow antigens to be detected better than in ruptured cells. Use of denaturing or harsh adjuvants, e.g., Freud's adjuvant, may rupture the cells and therefore is discouraged. The immunogen may be administered multiple times at periodic intervals such as, bi-weekly, or weekly, or may be administered in such a way as to maintain viability in the animal (e.g., in a tissue recombinant). Hybridomas are prepared from fusing splenocytes and a murine tumor partner, using, for example, the general somatic cell hybridization technique (Kohler and Milstein (1975) Nature 256:495-497).

As another alternative to the cell fusion technique, EBV immortalized B cells may be used to produce monoclonal antibodies of the subject invention. The hybridomas are expanded and subcloned, if desired, and supernatants are assayed for anti-immunogen activity by conventional assay procedures (e.g., FACS, IHC, radioimmunoassay, enzyme immunoassay, fluorescence immunoassay, etc.).

In another alternative, the antibodies can be made recombinantly. Methods for making recombinant antibodies are well known in the art. Monoclonal antibody mKID2 and any other equivalent antibodies can be sequenced and produced recombinantly in vitro. In one embodiment, mKID2 is sequenced and the polynucleotide sequence is then cloned into a vector for expression or propagation. The sequence encoding the antibody of interest may be maintained in a vector in a host cell and the host cell can then be expanded and frozen for future use. In another alternative, antibodies may be made recombinantly by phage display technology. See, for example, U.S. Pat. Nos. 5,565,332; 5,580,717; 5,733,743; 6,265,150; and Winter et al., Annu. Rev. Immunol. (1994) 12:433-455.

The antibody mKID2 or any other antibodies or protein of interest may be subjected to sequencing by Edman degradation, which is well-known to those of skill in the art. The peptide information generated from mass spectrometry or Edman degradation can be used to design probes or primers that are used to clone the protein of interest.

An alternative method of cloning the protein of interest is by “panning” using ALCAM for cells expressing the antibody or protein of interest. The “panning” procedure is conducted by obtaining a cDNA library from tissues or cells that express the antibody or protein of interest, over-expressing the cDNAs in a second cell type, and screening the transfected cells of the second cell type for a specific binding to ALCAM. Detailed descriptions of the methods used in cloning mammalian genes coding for cell surface proteins by “panning” can be found in the art. See, for example, Aruffo, A. and Seed, B. Proc. Natl. Acad. Sci. USA, 84, 8573-8577 (1987) and Stephan, J. et al., Endocrinology 140: 5841-5854 (1999).

cDNAs can be obtained by reverse transcribing the mRNAs from a particular cell type according to standard methods in the art. Specifically, mRNA can be isolated using various lytic enzymes or chemical solutions according to the procedures set forth in Sambrook, et al. supra or extracted by commercially available nucleic-acid-binding resins following the accompanying instructions provided by manufacturers (e.g., Qiagen, Invitrogen, Promega). The synthesized cDNAs are then introduced into an expression vector to produce the antibody or protein of interest in cells of a second type. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, and cosmids.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. Preferably, the host cells express the cDNAs at, a level of about 5 fold higher, more preferably 10 fold higher, even more preferably 20 fold higher than that of the corresponding endogenous antibody or protein of interest, if present, in the host cells. Screening the host cells for a specific binding to ALCAM is effected by an immunoassay or FACS. A cell overexpressed the antibody or protein of interest can be identified.

The invention includes polypeptides comprising an amino acid sequence of the antibodies of this invention, such as mKID2. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, a mKID2 polypeptide could be produced by an automated polypeptide synthesizer employing the solid phase method.

The invention also encompasses single chain variable region fragments (“scFv”) of antibodies of this invention, such as mKID2. Single chain variable region fragments are made by linking light and/or heavy chain variable regions by using a short linking peptide. Bird et al. (1988) Science 242: 423-426. An example of a linking peptide is (GGGGS)₃ (SEQ ID NO:1), which bridges approximately 3.5 nm between the carboxy terminus of one variable region and the amino terminus of the other variable region. Linkers of other sequences have been designed and used. Bird et al. (1988). Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The invention includes modifications to antibodies, such as antibody mKID2 and other anti-ALCAM antibodies, including functionally equivalent antibodies and polypeptides that do not significantly affect their properties and variants that have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs. Amino acid residues which can be conservatively substituted for one another include but are not limited to: glycine/alanine; valine/isoleucine/leucine; asparagine/glutamine; aspartic acid/glutamic acid; serine/threonine; lysine/arginine; and phenylalanine/tryosine. These polypeptides also include glycosylated and nonglycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the variable region. Changes in the variable region can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified mKID2 polypeptides are made using established procedures in the art and can be screened using standard assays known in the art.

The invention also encompasses fusion proteins comprising one or more fragments or regions from the antibodies of this invention, such as mKID2. In one embodiment, a fusion polypeptide is provided that comprises at least 10 contiguous amino acids of variable light chain region and at least 10 amino acids of variable heavy chain region. In another embodiment, the fusion polypeptide.contains a heterologous immunoglobulin constant region. In another embodiment, the fusion polypeptide contains a light chain variable region and a heavy chain variable region of mKID2. For purposes of this invention, a mKID2 fuision protein contains one or more mKID2 polypeptides and another amino acid sequence to which it is not attached in the native molecule, for example, a heterologous sequence or a homologous sequence from another region. A mKID2 polypeptide can be created by methods known in the art, for example, synthetically or recombinantly.

Since the antibodies generated are typically murine or other non-human antibodies, the antibodies produced from the hybridomas elicit an immune response in human individuals. To reduce the immune response, antibodies can be “humanized”. Techniques for the production of humanized antibodies are known in the art. A chimeric molecule that contains variable regions from the mouse anti-ALCAM antibody and constant regions from a human immunoglobulin can be produced. The immune response may be further minimized by grafting only the mouse antibody complimentary determining regions (CDRs) onto a human antibody variable framework, but this modification can lead to a diminution of binding activity in the resulting chimeric antibody. A further improvement can involve the optimization of antibody variable regions by identification of an altered antibody variable region with an enhanced binding affinity (See for example WO 01/27160).

In some embodiments, mKID2 chimeras are provided in which the heavy and/or light chains are fusion proteins. In some embodiments, the constant domain of the chains is from one particular species and/or class, and the variable domains are from a different species and/or class. For instance, a chimeric antibody (in some embodiments) is one in which the constant regions are derived from human origin, and the variable regions are homologous or derived from mKID2 (i.e., murine). Also embodied within the invention is an antibody with a humanized variable region, in which (in some embodiments) the CDR regions comprise mKID2 amino acid sequences, while the framework regions are derived from human sequences. Other forms of humanized antibodies are known in the art and described herein. Also embodied are functional fragments of chimeras. An example is a humanized Fab fragment, which contains a human hinge region, a human first constant region, a human kappa light or heavy chain constant region, and the variable region of light and/or heavy chain from mKID2. The humanized mKID2 Fab fragments can in turn be made to form Fab dimers. Typically, the mKID2 fusion proteins and mKID2 chimeras of this invention are made by preparing an expressing a polynucleotide encoding them using recombinant methods described herein, although they may also be prepared by other means known in the art, including, for example, chemical synthesis. See, for example, U.S. Pat. Nos. 5,807,715; 4,816,567; and 6,331,415.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. For example, the constant region may be engineered to more resemble human constant regions to avoid immune response if the antibody is used in clinical trials and treatments in humans. See, for example, U.S. Pat. Nos. 5,997,867 and 5,866,692.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J Immunol. 138:4534-4538 (1987), and Brown et al. Cancer Res. 47:3577-3583 (1987). Other references describe rodent CDRs grafted into a human supporting framework region (FR) prior to fusion with an appropriate human antibody constant domain. See, for example, Riechmann et al. Nature 332:323-327 (1988), Verhoeyen et al. Science 239:1534-1536 (1988), and Jones et al. Nature 321:522-525 (1986). Another reference describes rodent CDRs supported by recombinantly veneered rodent framework regions. See, for example, European Patent Publication No. 519,596. These “humanized” molecules are designed to minimize unwanted immunological response toward rodent anti-human antibody molecules, which limits the duration and effectiveness of therapeutic applications of those moieties in human recipients. Other methods of humanizing antibodies that may also be utilized are disclosed by Daugherty et al., Nucl. Acids Res., 19:2471-2476 (1991) and in U.S. Pat. Nos. 6,180,377; 6,054,297; 5,997,867; 5,866,692; 6,210,671; 6,350,861; and PCT WO 01/27160.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

V. Methods for Screening Antibodies that Bind to ALCAM

Several methods may be used to screen monoclonal antibodies that bind to ALCAM. One method that may be employed is immunohistochemistry (IHC). Standard immunohistochemical techniques are known to those of skill in the art. See, for example, Animal Cell Culture Methods (J. P. Mather and D. Barnes, eds., Academic Press, Vol. 57, Ch. 18 and 19, pp. 314-350, 1998).

The first step in IHC screening to select for an appropriate anti-ALCAM antibody is the binding of primary antibodies to various tissues or cells. Biological samples (e.g., tissues) may be obtained from biopsies, autopsies, or necropsies. In one embodiment, the tissue sample is sections of frozen tissue from different organs. Frozen tissues can be prepared, sectioned, with or without fixation, and IHC performed by any of a number of methods known to one familiar with the art. See, for example, Stephan et al. Dev. Biol. 212: 264-277 (1999), and Stephan et al. Endocrinology 140: 5841-54 (1999). In another embodiment, the tissue sample is sections of fresh tissue. The cells or tissue samples can be either cancerous or non-cancerous.

Several different detection systems may be utilized to detect binding of antibodies to tissue section. Typically, immunohistochemistry involves the binding of a primary antibody to the tissue and then following with a secondary antibody reactive against the species from the primary antibody conjugated to a detectable marker (e.g., horseradish peroxidase, HRP, or alkaline phosphotase). One alternative method that may be used is polyclonal mirror image complementary antibodies or polyMICA. PolyMICA (polyclonal Mirror Image Complementary Antibodies) technique, described; by D. C. Mangham and P. G. Isaacson (Histopathology (1999) 35(2):129-33), can be used to test binding of primary antibodies to normal and cancerous tissue. Several kinds of polyMICA™ Detection kits are commercially available from The Binding Site Limited (P.O. Box 4073 Birmingham B29 6AT England). Product No. HK004.D is a polyMICA™ Detection kit that uses DAB chromogen. Product No. HK004.A is a polyMICA™ Detection kit that uses AEC chromogen. Alternatively, the primary antibody may be directly labeled with the detectable marker.

To ascertain if an antibody binds specifically to ALCAM, the antibody may be assayed for binding ALCAM in a Western blot (see Example 1). Alternatively, specific binding of an antibody to ALCAM may be assayed by binding on tissue known to express ALCAM. Tissue samples can be embedded in a solid or semi-solid substance that prevents damage during freezing (e.g., agarose gel or OCT) and then sectioned for staining. Cancers from different organs and at different grades can be used to screen antibodies. Examples of tissues that may be used for screening purposes include but are not limited to ovary, lung, prostate, pancreas, colon, and breast.

In yet another alternative, cancerous cell lines such as, but not limited to, SK-Ov-3 (ATCC #HTB 77), CFPAC-1 (ATCC #CRL 1918), and HPAF-II (ATCC #CRL-1997) and normal cells such as, but not limited to, ovarian epithelial, bronchial epithelial, and activated leukocytes from their respective tissues may be used to screen for monoclonal antibodies which have a binding affinity for ALCAM. Cell cultures known to be void of ALCAM expression such as MOLT-3 (ATCC #CRL-1552) and SK-LMS-1 (ATCC #HTB 88) can be used as negative controls. The cancerous or non-cancerous cells can be grown on glass slides or coverslips, or on plastic surfaces, or prepared in a CellArray™, as described in WO 01/43869, and screened for the binding of antibody using IHC as described above for tissues. Alternatively, cells may be removed from the growth surface using non-proteolytic means and spun into a pellet that is then embedded and treated as tissues for IHC analysis as described above. In another alternative, single cells may be screened by incubating with the primary antibody, a secondary reporter antibody linked to a fluorescent molecule and then analyzed using a fluorescent activated cell sorting (FACS) machine.

Antibodies that bind to cancerous cells or tissues that express ALCAM are selected. In a preferred embodiment, the monoclonal antibody is cross-reactive with human cells. 2D4 and mKID2 are examples of antibodies that bind to the antigen ALCAM, which is present on a number of different cancer tissues, including but not limited to ovarian, lung, prostate, pancreas, colon, and breast tissue.

Monoclonal antibody-secreting hybridomas described above can be selected for producing antibodies that bind preferentially to the same epitopes on ALCAM that a known anti-ALCAM antibody preferentially binds. Methods of selecting such antibody are known in the art. For example, binding competition assays can be used to determine whether an antibody competitively inhibits binds to the same epitope as the original antibody. An antibody's competition with another antibody for binding to ALCAM indicates that the antibody binds preferentially to the same epitope that the original antibody binds. It is presently believed that there are at least two and likely more epitopes found on ALCAM as it presents on the cell surface in its native environment and native configuration. Therefore, anti-ALCAM antibodies that may have varying binding specificities yet still retain the desirable biological effects of the claimed invention are encompassed herein. Binding competition assays are well known in the art.

Epitope mapping may be used to further characterize the antibody. Commercially available services (e.g., Pepscan Systems, P.O. Box 2098, 8203 AB Lelystad, The Netherlands) may be used to determine the epitope(s) on the antigen ALCAM to which an antibody binds.

VI. Association of Anti-ALCAM Antibodies with Chemotherapeutic Agents

In one embodiment, an antibody (or fragment thereof) against ALCAM can be associated with (including linked to) a chemotherapeutic agent. The chemotherapeutic agents are administered to an individual in need of such treatment to deliver these agents to cancer cells expressing the antigen recognized by the antibody and thus eliminate cancerous cells. Chemotherapeutic agent include radioactive molecules, toxins, also referred to as cytotoxins or cytotoxic agents, which includes any agent that is detrimental to the viability of cancerous cells, agents, and liposomes or other vesicles containing chemotherapeutic compounds. Examples of chemotherapeutic agents include but are not limited to calicheamicin, maytansinoid, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and antimitotic agents (e.g., vincristine and vinblastine). In a preferred embodiment, the cytotoxin is especially effective in dividing or rapidly dividing cells, such that non-dividing cells are relatively spared from the toxic effects.

The antibodies of the invention can be internalized within the carcinoma cells (such as ovarian cancer cells) to which they bind and are therefore particularly useful for therapeutic applications, for example, delivering into the cells toxins that need to be internalized for their adverse activity. Examples of such toxins include, but not limited to, saporin, calicheamicin, auristatin, and maytansinoid.

The antibodies or polypeptides of the invention can be associated (including conjugated or linked) to a radioactive molecule, a toxin, or other therapeutic agents, or to liposomes or other vesicles containing therapeutic agents covalently or non-covalently, directly or indirectly. The antibody may be linked to the radioactive molecule, the toxin, or the chemotherapeutic molecule at any location along the antibody so long as the antibody is able to bind its target ALCAM.

A toxin or a chemotherapeutic agent may be coupled (e.g., covalently bonded) to a suitable monoclonal antibody either directly or indirectly (e.g., via a linker group, or, alternatively, via a linking molecule with appropriate attachment sites, such as a platform molecule as described in U.S. Pat. No. 5,552,391). The toxin and chemotherapeutic agent of the present invention can be coupled directly to the particular targeting proteins using methods known in the art. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

The antibodies or polypeptides can also be linked to a chemotherapeutic agent via a microcarrier. Microcarrier refers to a biodegradable or a non-biodegradable particle which is insoluble in water and which has a size of less than about 150, 120 or 100 μm in size, more commonly less than about 50-60 μm, preferably less than about 10, 5, 2.5, 2 or 1.5 μm. Microcarriers include “nanocarriers”, which are microcarriers have a size of less than about 1 μm, preferably less than about 500 nm. Such particles are known in the art. Solid phase microcarriers may be particles formed from biocompatible naturally occurring polymers, synthetic polymers or synthetic copolymers, which may include or exclude microcarriers formed from agarose or cross-linked agarose, as well as other biodegradable materials known in the art. Biodegradable solid phase microcarriers may be formed from polymers which are degradable (e.g., poly(lactic acid), poly(glycolic acid) and copolymers thereof) or erodible (e.g., poly(ortho esters such as 3,9-diethylidene-2,4,8,10-tetraoxaspiro[5.5]undecane (DETOSU) or poly(anhydrides), such as poly(anhydrides) of sebacic acid) under mammalian physiological conditions. Microcarriers may also be liquid phase (e.g., oil or lipid based), such liposomes, iscoms (immune-stimulating complexes, which are stable complexes of cholesterol, and phospholipid, adjuvant-active saponin) without antigen, or droplets or micelles found in oil-in-water or water-in-oil emulsions, provided the liquid phase microcarriers are biodegradable. Biodegradable liquid phase microcarriers typically incorporate a biodegradable oil, a number of which are known in the art, including squalene and vegetable oils. Microcarriers are typically spherical in shape, but microcarriers that deviate from spherical shape are also acceptable (e.g., elipsoid, rod-shaped, etc.). Due to their insoluble nature (with respect to water), microcarriers are filterable from water and water-based (aqueous) solutions.

The antibody or polypeptide conjugates of the present invention may include a bifunctional linker that contains both a group capable of coupling to a toxic agent or chemotherapeutic agent and a group capable of coupling to the antibody. A linker can function as a spacer to distance an antibody from an agent in order to avoid interference with binding capabilities. A linker can be cleavable or non-cleavable. A linker can also serve to increase the chemical reactivity of a substituent on an agent or an antibody, and thus increase the coupling efficiency. An increase in chemical reactivity may also facilitate the use of agents, or functional groups on agents, which otherwise would not be possible. The bifunctional linker can be coupled to the antibody by means that are known in the art. For example, a linker containing an active ester moiety, such as an N-hydroxysuccinimide ester, can be used for coupling to lysine residues in the antibody via an amide linkage. In another example, a linker containing a nucleophilic amine or hydrazine residue can be coupled to aldehyde groups produced by glycolytic oxidation of antibody carbohydrate residues. In addition to these direct methods of coupling, the linker can be indirectly coupled to the antibody. by means of an intermediate carrier such as an aminodextran. In these embodiments the modified linkage is via either lysine, carbohydrate, or an intermediate carrier. In one embodiment, the linker is coupled site-selectively to free thiol residues in the protein. Moieties that are suitable for selective coupling to thiol groups on proteins are well known in the art. Examples include disulfide compounds, α-halocarbonyl and α-halocarboxyl compounds, and maleimides. When a nucleophilic amine function is present in the same molecule as an α-halo carbonyl or carboxyl group the potential exists for cyclization to occur via intramolecular alkylation of the amine. Methods to prevent this problem are well known to one of ordinary skill in the art, for example by preparation of molecules in which the amine and α-halo functions are separated by inflexible groups, such as aryl groups or trans-alkenes, that make the undesired cyclization stereochemically disfavored. See, for example, U.S. Pat. No. 6,441,163 for preparation of conjugates of maytansinoids and antibody via a disulfide moiety.

One of the cleavable linkers that can be used for the preparation of antibody-drug conjugates is an acid-labile linker based on cis-aconitic acid that takes advantage of the acidic environment of different intracellular compartments such as the endosomes encountered during receptor mediated endocytosis and the lysosomes. See, for example, Shen et al., Biochem. Biophys. Res. Commun. 102:1048-1054 (1981) for the preparation of conjugates of daunorubicin with macromolecular carriers; Yang et al., J. Natl. Canc. Inst. 80:1154-1159 (1988) for the preparation of conjugates of daunorubicin to an anti-melanoma antibody; Dillman et al., Cancer Res. 48:6097-6102 (1988) for using an acid-labile linker in a similar fashion to prepare conjugates of daunorubicin with an anti-T cell antibody; Trouet et al., Proc. Natl. Acad. Sci. 79:626-629 (1982) for linking daunorubicin to an antibody via a peptide spacer arm.

An antibody (or polypeptide) of this invention may be conjugated (linked) to a radioactive molecule by any method known to the art. For a discussion of methods for radiolabeling antibody see “Cancer Therapy with Monoclonal AntibodiesT”, D. M. Goldenberg ed. (CRC Press, Boca Raton, 1995).

Alternatively, an antibody can be conjugated to a second antibody to form an antibody heteroconjugate as described by Segal in U.S. Pat. No. 4,676,980. The formation of cross-linked antibodies can target the immune system to specific types of cells, for example, cancer cells expressing ALCAM.

VII. ALCAM is a Marker for a Variety of Non-Melanoma Cancers

ALCAM has been disclosed to be expressed in metastasizing malignant melanoma and not detectable in non-metastasizing cell lines (Degen et al., (1998) Am J Pathol 152:805-813). The anti-ALCAM monoclonal antibody 2D4 and mKID2 were used to screen a variety of non-melanoma cancer cells, including ovarian, lung, prostate, pancreatic, colon, and breast cancer cells. We have discovered that certain cancer cells, other than metastatic malignant melanoma, express ALCAM, including ovarian, lung, prostate, pancreatic, colon, and breast cancer cells. The use of anti-ALCAM antibodies to screen for cancer cells is dependent upon differential expression of ALCAM on cancer cells versus normal cells of the same cell type. In one embodiment, the methods include contacting a biological sample with an anti-ALCAM antibody, and comparing the presence or absence of ALCAM in a test sample with the presence or absence of ALCAM in a control sample. Primary and metastasizing ovarian and prostate cancer tissues were screened with the anti-ALCAM monoclonal antibody 2D4 and found to express ALCAM in cells that are negative for ALCAM expression in normal tissue. The expression levels of ALCAM in ovarian and prostate cancer tissue, as well as a panel of normal tissues, using the anti-ALCAM monoclonal antibody, 2D4, generated by transgenic mice supplied by Medarex, Inc. (Princeton, N.J.), is shown in Example 2. Primary and metastasizing pancreatic cancer cell lines were also screened for expression of ALCAM. As shown in Example 3, eight of nine pancreatic cancer cell lines express detectable levels of ALCAM whereas ALCAM only expressed in ductal epithelial cells in normal pancreas. A primary squamous carcinoma lung cancer cell line was also screened and found to express ALCAM, as shown in Example 4. Expressions of ALCAM in colon, lung, prostate, and breast cancer tissue, and skin, kidney, lung, liver, pancreas, colon, and duodenum normal tissue were also screened using monoclonal antibody mKID2, as shown in Example 6.

VIII. Methods of Diagnosing Cancer

Monoclonal antibodies made by the methods disclosed herein may be used to identify or detect the presence or absence of cancer cells that express the antigen ALCAM in a variety of cells and tissues, including but not limited to ovary, lung, prostate, pancreatic, colon, or breast cells and tissues, for purposes of diagnosis. This can involve the formation of a complex between ALCAM and an antibody that binds specifically to ALCAM to evaluate the level of ALCAM in a biological sample. In a preferred embodiment, the antibody bears a detectable label. Examples of labels that may be used include a radioactive agent or a fluorophore, such as fluoroisothiocyanate or phycoerythrin. The formation of such a complex can be in vitro or in vivo. The monoclonal antibodies may also be used to identify cancerous cells at different stages of development. The antibodies may recognize both primary and metastasizing cancers of the ovary, prostate and pancreas and primary cancers of the lung that express ALCAM. As used herein, detection may include qualitative and/or quantitative detection and may include comparing the level measured to a normal cell for an increased level of expression of ALCAM in cancerous cells.

The invention also provides methods of aiding diagnosis of cancer (such as ovarian, lung, pancreatic, prostate, colon, or breast cancer) in an individual using any antibody that binds to ALCAM and any other methods that can be used determine the level of ALCAM expression. As used herein, methods for “aiding diagnosis” means that these methods assist in making a clinical determination regarding the classification, or nature, of cancer, and may or may not be conclusive with respect to the definitive diagnosis. Accordingly, a method of aiding diagnosis of cancer can comprise the step of detecting the level of ALCAM in a biological sample from the individual and/or determining the level of ALCAM expression in the sample.

The anti-ALCAM antibodies made by the methods disclosed herein may also be used to determine whether an individual diagnosed with cancer may be deemed a candidate for immunotherapy using antibodies directed against ALCAM. In one embodiment, a cancerous tumor or a biopsy sample may be tested for expression of ALCAM, using antibodies directed against ALCAM. Individuals with cancer cells that express ALCAM are suitable candidates for immunotherapy using antibodies directed against ALCAM. Methods of using an antibody against ALCAM for immunotherapy candidate screening purposes is useful both before and after any form of anti-cancer treatment, e.g., chemotherapy or radiation therapy, to determine which tumors are most likely to respond to a given treatment, individual prognosis, tumor subtype or origin of metastatic disease, and progression of the disease or response to treatment.

In vitro techniques for detection of ALCAM are routine in the art and include enzyme linked immunosorbent assays (ELISAs), immunoprecipitations, immunofluorescence, enzyme immunoassay (EIA), radioimmunoassay (RIA), and Western blot analysis. In one embodiment, tissues with the cancerous cells are removed and the tissues are prepared for immunohistochemistry by methods well known in the art, e.g., embedding in a freezing compound, freezing and sectioning, with or without fixation; fixation and paraffin embedding with or without various methods of antigen retrieval and counterstaining.

In another embodiment, ALCAM expression is detected in non-melanoma cancer cells using the monoclonal antibody 2D4, mKID2, or any other ALCAM binding embodiment described herein. For simplicity, reference will be made generally to 2D4 or mKID2 with the understanding that these methods apply to any of the ALCAM binding embodiments described herein. The anti-ALCAM monoclonal antibody 2D4 and mKID2, specifically binds to various cancer cell types that express ALCAM, including but not limited to ovarian, lung, prostate, pancreatic, colon, and breast cancer cells. Not all cells in the ovary, lung, prostate, pancreas, colon, or breast cancer cells express ALCAM, and cancerous cells in other tissues may express ALCAM, thus an individual should be screened for the presence or absence of ALCAM on cancerous cells to determine the usefulness of immunotherapy in the individual. In one embodiment, normal prostate epithelial cells may be differentiated from cancerous prostate epithelial cells by staining prostate tissue with anti-ALCAM antibodies. Normal prostate epithelial cells are histologically well organized while cancerous tissues have a very different morphology. Staining with anti-ALCAM antibody may be used to distinguish cancerous tissues from normal tissues.

Labeled anti-ALCAM monoclonal antibody 2D4 was used to screen a panel of pancreatic cancer cell lines. As shown in Example 3, the anti-ALCAM antibody 2D4 binds to cancerous pancreatic cell lines of multiple lineage, with eight of nine pancreatic cancer cell lines expressing ALCAM. The monoclonal antibody 2D4 reacts with both metastatic and primary pancreatic cancer cell lines.

In another embodiment, monoclonal antibodies, other than monoclonal antibody 2D4, that bind specifically to ALCAM may be used to screen for the presence of ALCAM in non-melanoma cancer cells and tissues.

One in vivo technique for detection of ALCAM that can be used is introducing a labeled anti-ALCAM antibody into an individual who has been diagnosed with cancer. In one embodiment, a method of using the antibodies for immunotherapy candidate screening is in vivo tumor imaging by linking the antibody to a radioactive or radioopaque agent, administering the antibody to the individual and using an x-ray or other imaging machine to visualize the localization of the labeled antibody to a location of known cancer cells (e.g. a tumor). In some embodiments, the anti-ALCAM antibody is mKID2 or biologically equivalent antibodies or polypeptides that bind to ALCAM.

Antibodies (or polypeptides) recognizing the antigen may also be used to create diagnostic immunoassays for detecting antigen released or secreted from living or dying cancer cells in bodily fluids, including but not limited to, blood, saliva, urine, pulmonary fluid, or ascites fluid. As discussed in further detail in the Examples, 2D4 and mKID2 can bind to various forms cancer in different stages from tissues including but not limited to ovary, breast, lung, prostate, colon, and pancreas. Methods of using the antibodies and agents of this invention for diagnostic purposes is useful both before and after any form of anti-cancer treatment, e.g., chemotherapy or radiation therapy, to determine which tumors are most likely to respond to a given treatment, patient prognosis, tumor subtype or origin of metastatic disease, and progression of the disease or response to treatment.

IX. Methods of Using Antibodies Against ALCAM for Immunotherapy

The monoclonal antibody, 2D4, and other anti-ALCAM antibodies (such as humanized or chimeric antibodies) made by the methods disclosed herein may be used, for example, for therapeutic purposes in individuals with cancer cells that express ALCAM, including ovary, lung, prostate, pancreas, colon, or breast cancer cells. Therapy can involve formation of complexes of anti-ALCAM antibody and ALCAM both in vitro and/or in vivo as described above. In a preferred embodiment, therapy with antibodies against ALCAM can involve the association of an anti-ALCAM antibody with a chemotherapeutic agent or another antibody, as described above.

The invention provides methods of delivering any of the compositions (including conjugates) described herein to ALCAM expressing cells, such as ALCAM expression cancer cells. These methods entail administering the compositions (including conjugates) described herein to an individual. In some embodiments, the methods provide for introducing, for example, a conjugate into a target cell. In yet another embodiment, an anti-ALCAM antibody (such as a humanized or chimeric form of an anti-ALCAM antibody and various fomulations of those antibodies) can be conjugated (including linked) to a chemotherapeutic agent (such as a radioactive molecule, a toxin, e.g., saporin, calicheamicin, auristatin, or maytansinoid, or other chemotherapeutic molecule) or to liposomes or other vesicles containing chemotherapeutic compounds and administered to an individual to target these compounds to the cancer cell containing the antigen recognized by the antibody and thus eliminate cancerous cells. In some embodiments, the chemotherapeutic agent is delivered into cancerous cells (such as ovarian cancerous cells).

This invention also provides methods of inhibiting growth and/or proliferation of prostate, lung, breast, ovarian, pancreatic, or colon cancer cells using an anti-ALCAM antibody or other embodiments that bind to ALCAM linked to a chemotherapeutic agent. In some embodiments, the antibody is a humanized or chimeric form of a non-human anti-ALCAM antibody.

This invention also provides methods of delaying development of metastasis in an individual with cancer (including, but not limited to, prostate, lung, breast, ovarian, pancreatic, or colon cancer) using an anti-ALCAM antibody or other embodiments that bind to ALCAM linked to a chemotherapeutic agent. In some embodiments, the antibody is a humanized or chimeric form of a non-human anti-ALCAM antibody.

In yet another embodiment, the antibody can be employed as adjuvant therapy at the time of the surgical removal of a cancer expressing the antigen in order to delay the development of metastasis. The antibody or antibody associated with a chemotherapeutic agent can also be administered before surgery (neoadjuvant therapy) in an individual with a tumor expressing the antigen in order to decrease the size of the tumor and thus enable or simplify surgery, spare tissue during surgery, and/or decrease the resulting disfigurement.

In yet another embodiment, mKID2 or any of the ALCAM binding embodiments described herein can bind to ALCAM expressing cancerous cells and induces an active immune response against the cancerous cells expressing ALCAM. In some cases, the active immune response can cause the death of the cancerous cells (e.g., antibody binding to cancer cells inducing apoptotic cell death), or inhibit the growth (e.g., block cells cycle progression) of the cancerous cells. In other cases, mKID2 or any of the antibodies described herein can bind to cancerous cells and antibody dependent cellular cytotoxicity (ADCC) can eliminate cancerous cells to which mKID2 binds. Accordingly, the invention provides methods of stimulating an immune response comprising administering any of the compositions described herein.

In some cases, antibody binding can also activate both cellular and humoral immune responses and recruit more natural killer cells or increased production of cytokines (e.g., IL-2, IFN-γ, IL-12, TNF-α, TNF-β, etc.) that further activate an individual's immune system to destroy cancerous cells. In yet another embodiment, mKID2 can bind to cancerous cells, and macrophages or other phagocytic cell can opsonize the cancerous cells.

Various formulations of an antibody against ALCAM or fragments (e.g., Fab, Fab′, F(ab′)₂, Fv, Fc, etc.), such as chimeric antibodies, single chain (ScFv), mutants thereof, fusion proteins comprising an antibody portion, humanized antibodies, and any other modified configuration of an anti-ALCAM antibody that comprises an antigen ALCAM recognition site of the required specificity, that are associated (including linked to) a chemotherapeutic agent, may be used for administration. In some embodiments, an antibody against ALCAM or fragments thereof may be administered undiluted. In other embodiments, an antibody against ALCAM or fragments thereof and a pharmaceutically acceptable excipient are administered, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington: The Science and Practice of Pharmacy, 20th edition, Lippincott, Williams & Wilkins, Publishing.

Generally, these agents are formulated for administration by injection (e.g., intraperitoneally, intravenously, subcutaneously, intramuscularly, etc.), although other forms of administration (e.g., oral, mucosal, etc) can be also used. Accordingly, an antibody against ALCAM and equivalents thereof are preferably combined with pharmaceutically acceptable excipients such as saline, Ringer's solution, dextrose solution, and the like. The particular dosage regimen, i.e., dose, timing and repetition, will depend on the particular individual and that individual's medical history. Generally, any of the following doses may be used: a dose of at least about 50 mg/kg body weight; at least about 10 mg/kg body weight; at least about 3 mg/kg body weight; at least about 1 mg/kg body weight; at least about 750 microg/kg body weight; at least about 500 microg/kg body weight; at least about 250 microg/kg body weight; at least about 100 microg/kg body weight; at least about 50 microg/kg body weight; at least about 10 microg /kg body weight; at least about 1 microg/kg body weight, or more, is administered. Empirical considerations, such as the half life, generally will contribute to determination of the dosage. Antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is based on reducing the number of cancerous cells, maintaining the reduction of cancerous cells, reducing the proliferation of cancerous cells, or delaying the development of metastasis. Alternatively, sustained continuous release formulations of antibodies against ALCAM may be appropriate. Various formulations and devices for achieving sustained release are known in the art.

In one embodiment, dosages for an antibody against ALCAM may be determined empirically in individuals who have been given one or more administration(s). Individuals are given incremental dosages of an antibody against ALCAM. To assess efficacy of the antibody against ALCAM or fragments thereof, the specific cancer disease state can be followed by methods such as direct measurement of tumor size via palpation or visual observation, indirect measurement of tumor size by x-ray or other imaging techniques, an improvement as assessed by direct tumor biopsy and microscopic examination of the tumor sample, the measurement of an indirect tumor marker (e.g., PSA for prostate cancer), a decrease in pain, paralysis, impairment of speech, vision, breathing or other disability associated with the tumor, increased appetite, or an increase in quality of life as measured by accepted tests or prolongation of survival. It will be apparent to one of skill in the art that the dosage will vary depending on the individual, the type of cancer, the stage of cancer, whether the cancer has begun to metastasize to other location in the individual, and the past and concurrent treatments being used.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

In some embodiments, more than one antibody maybe present. Such compositions may contain one or more than one antibody (may contain at least one, at least two, at least three, at least four, at least five different antibodies) that is reactive against, for example, ovarian, lung, prostate, pancreatic, colon, or breast cancer cells. A mixture of antibodies, as they are often denoted in the art, may be particularly useful in treating a broader range of population of individuals.

Assessment of disease is performed using standard methods in the arts, such as imaging methods and monitoring appropriate marker(s).

X. ALCAM is Involved in Neo-Vascularization$

Cells or tissue that express ALCAM on the cell surface, including adult cells and tissue and grafted cells and tissue, activate neovascularization upon administration of anti-ALCAM antibodies. As shown in Example 5, a proprietary human pancreatic tumor cell line expressing ALCAM, Rav 9926, was planted into nude mice. Administration of the anti-ALCAM monoclonal antibody, 2D4, resulted in neovascularization of the human tumor with mouse blood vessels. In further experiments, SKOV-3, a human ovarian cancer cell line which expresses ALCAM, was grown under the kidney capsule or as a subcutaneous tumor. Administration of 2D4 resulted in increased neovascularization of the resulting ovarian tumors. Since the monoclonal antibody 2D4 does not bind to mouse ALCAM, MAb 2D4 bound to and activated foreign cells expressing ALCAM (i.e., human pancreatic tumor cells) and triggered blood vessel growth. Without being bound by theory, activation of ALCAM results in the release of a vascularization-promoting molecule that promotes blood vessel generation.

Accordingly, in one embodiment, anti-ALCAM agents, such as an anti-ALCAM antibody, may be administered to an individual in an amount effective to trigger blood vessel growth in tissue. In one embodiment, the individual has received a tissue transplant and may be in need of vascularization of the tissue transplant. Administration of an effective amount of an anti-ALCAM agent can be used to achieve neovascularization.

In another embodiment, anti-ALCAM antibodies may be used to identify a vascularization-promoting molecule. For example, anti-ALCAM antibodies can be used to stimulate cells to produce neovascularization promoting activity. A molecule that promotes neovascularization or vascularization can be purified by standard techniques, such as affinity chromatography on cell lysates from cells that exhibit such activity. Polynucleotides (e.g. DNA or RNA) encoding such activities can be isolated and cloned using standard techniques such as expression cloning, differential display, and gene array. In one embodiment, the invention may identify a vascularization-promoting molecule by stimulating tumor cells with an anti-ALCAM antibody, fractionating the molecules released from the tumor cells, and analyzing each fraction for vascularization activity to identify a vascularization-promoting molecule that is released from tumor cells upon stimulation by an anti-ALCAM antibody.

In another embodiment, a vascularization-promoting molecule may be administered to an individual in an amount effective to trigger blood vessel growth in tissue. An antibody against a vascularization-promoting molecule can be prepared by standard techniques known in the art, as described supra. In another embodiment, an agent, such as an antibody against an identified vascularization-promoting molecule may be administered to an individual in an amount effective to inhibit the neovascularization activity of the vascularization-promoting molecule.

An animal model, such as that described in Example 5, may be used to screen for one or more antagonist agents, such as an antagonist antibody, that inhibit the neovascularization activity of ALCAM. Following administration of a candidate antagonist agent, such as an antagonist antibody, the animal model is monitored for diminished neovascularization. A reduction in the amount of neovascularization would indicate that the candidate antibody has an inhibitory effect on blood vessel growth.

Anti-CD6 antibodies that have the desired characteristics described herein may be utilized in the practice of this invention. Additionally, certain CD6 peptides and polypeptide fragments and analog molecules are included within the scope of this invention, preferably those which share a common biological activity with the claimed ALCAM agonists and antagonists, including but not limited to those peptides which modulate the vascularization-related activities of ALCAM.

While this disclosure frequently discuses antibodies as candidate therapeutic agents of this invention, the compositions encompassed by this invention also include non-antibody agents that enhance or reduce ALCAM-mediated neovascularization. Without being limited to any specific mechanism of action, these neovascularization modulators may act directly on ALCAM or act on its native or induced binding partners. CD6 is an example of a known binding partner that is suitable for modulation with agents that enhance or reduce ALCAM-mediated neovascularization, according to the teachings of this invention. Agents can be tested for their ability to block, reduce, enhance or otherwise modulate the association of ALCAM with a binding partner, such as an anti-ALCAM antibody or CD6. Specifically, an agent can be tested for the ability to modulate such an interaction by incubating a peptide comprising the ALCAM interaction site (typically in its native conformation as it exists on intact living cells) with a binding partner and a test agent, and determining whether the test agent reduces or enhances the binding of the binding partner to the ALCAM peptide. Agonists, antagonists, and other modulators are expressly contemplated.

XI. Kits Comprising Antibodies which Bind to ALCAM

The invention also provides kits comprising antibodies that bind to ALCAM for use in screening individuals as candidates to receive immunotherapy of various cancers, neovascularization or inhibition of neovascularization of ALCAM expressing tissues. Accordingly, the kits comprise an antibody that can bind to ALCAM specifically and/or form a complex with ALCAM (useful, for example, for detecting ovarian, prostate, pancreatic, lung, colon, or breast cancerous cells). In some embodiments, the kits comprise antibody mKID2 or an antibody that competitively inhibits preferential binding of mKID2 to ALCAM, used as a single agent or linked to a chemotherapeutic agent or a labeling agent. These kits may further include instruction and/or reagents for linking the antibody or any antibody or polypeptide embodiments described herein to the chemotherapeutic agent(s) or the labeling agent(s). In some aspects, the binding of an antibody (e.g., monoclonal, human, humanized, etc.) is used for screening whether an individual would be a candidate for immunotherapy. In other aspects, the kits may be used, for example, to treat an individual with cancer. In some embodiments, kits for treating individual with cancer include kits for delivering a chemotherapeutic agent to cancerous cells (such as but not limited to ovarian, prostate, pancreatic, lung, colon, or breast cancerous cells), or for delivering a chemotherapeutic agent into cancerous cells (such as but not limited to ovarian cancerous cells). The kits of this invention are in suitable packaging, and may optionally provide additional components such as buffers and instructions for determining binding to ALCAM, such as capture reagents, developing reagents, labels, reacting surfaces, means for detection, control samples, and interpretive information. The instructions may be for any measurement of antigen binding, including, but not limited to, those assays described herein. In some embodiments, reagents described above are supplied such that multiple measurements may be made, such as allowing for measurements in the same individual over time or multiple individuals. Any appropriate means for detecting binding of the antibodies may be employed (and provided in the kits) such as a labeled anti-human antibody, wherein the label may be an enzyme, fluorophore, chemiluminescent material, radioisotope or coenzyme. In other embodiments, the anti-ALCAM antibody may be associated with a chemotherapeutic agent for use as a chemotherapeutic for treatment of an individual with cancer.

The following examples are provided to illustrate, but not to limit, the invention.

### EXAMPLES

### Example 1

Western Blot Analysis of ALCAM in Prostate Cancer Cells

LNCAP cells (prostate carcinoma cells (ATCC#CRL-1740 or CRL-10995) were grown to confluency on 175 cm² culture dishes. The confluent monolayer was washed three times with Hank's Balanced Salt Solution (HBSS+, containing no sodium bicarbonate, or phenol red; buffered with 10 mM HEPES, pH 7.4, obtained from Sigma Chemicals) and biotinylated with 200 microg of sulfo-NHS-LC-biotin (Pierce Endogen) for 30 minutes at room temperature. The cells were further washed three times with HBSS+ containing 0.1M Tris, pH 7.4 (Sigma Chemicals) and incubated in HBSS+ containing 0.1M Tris, pH 7.4 for 15 minutes at room temperature. The cells were finally washed three times with HBSS+ and lysed by incubation for 5 minutes, on ice, in lysis buffer (HBSS+ containing 2% Triton X-100, 2 mM PMSF, 0.1% sodium azide, and 1 tablet per 5 ml lysis buffer of EDTA free complete mini-protease cocktail, EDTA free complete mini-protease cocktail obtained from Roche Molecular Biochemicals, all else from Sigma Chemicals). Cells were scraped in lysis buffer and the lysates collected. Lysate was clarified by centrifugation, 14,000×g for one hour at 4 degrees C. Clarified lysate was first pre-cleared for 2 hours at 4 degrees C. with 5 microl of human IgG conjugated (1 mg/ml) CNBr 4MB sepharose beads (Amersham Pharmacia). Human IgG beads were removed, and the pre-cleared lysate was then incubated with monoclonal antibody 2D4 conjugated with CNBr 4MB sepharose beads (conjugated at 1 mg/ml) for 2 hours at 4 degrees C. The 2D4 beads were removed after the 2 hour incubation. Both the human IgG beads and the 2D4 beads were individually washed three times with 1 ml of lysis buffer, followed by three 1 ml washes with HBSS+. Washed beads were eluted by the addition of 30 microl of SDS-PAGE sample buffer and boiling at 99 degrees C. for 5 minutes. Similarly, ALCAM was immunoprecipitated from the LNCAP cell lysate by using anti-ALCAM monoclonal antibody clone 3A6 from Ancell Corporation, Minnesota. The samples were resolved on a 4-20% Novex gradient gel (Invitrogen), transferred onto 0.2 microm nitrocellulose (Invitrogen) and visualized by horse radish peroxidase (HRP) conjugated streptavidin (Pierce Endogen) or western blotted with 5 microg/blot of 2D4. For detection with HRP conjugated streptavidin, the nitrocellulose was first blocked for 1 hour with blocking buffer (5% non-fat dry milk in phosphate buffered saline containing 0.05% Tween-20 (PBST), Sigma Chemicals). HRP conjugated streptavidin was diluted into PBST at 1 microg/ml and exposed to the nitrocellulose for 30 minutes at room temperature. The nitrocellulose was washed three times with PBST before color development with DAB substrate. For western blotting with 2D4, the nitrocellulose was similarly blocked for 1 hour in blocking buffer. The nitrocellulose was then incubated in a heat sealed plastic pouch containing 1 ml of 5 microg/ml 2D4 diluted in blocking buffer. The nitrocellulose was washed 3 times with PBST before incubation with 10 ml of 1 microg/ml HRP conjugated donkey anti-human IgG (heavy and light chain specific, cross adsorbed against bovine, chicken, goat, guinea pig, syrian hamsters, horse, human, rabbit, sheep serum proteins, Jackson Immunoreasearch, Cat #709-035-149) for 1 hour at room temperature. The nitrocellulose was finally washed three times with PBST and visualized by color development using DAB substrate. For western blotting with polyclonal anti-ALCAM from Santa Cruz (sc-8548 and sc-8549), the nitrocellulose was similarly blocked for 1 hour in blocking buffer. The nitrocellulose was then incubated in a heat sealed plastic pouch containing 1 ml primary antibody diluted in blocking buffer according to supplier's recommendation. The nitrocellulose was washed 3 times with PBST before incubation with 10 ml of 1 microg/ml HRP conjugated donkey anti-goat IgG (heavy and light chain specific, Jackson Immunoresearch, Cat #705-035-147) for 1 hour at room temperature. The nitrocellulose was finally washed three times with PBST and visualized by color development using DAB substrate

The results showed both 2D4 and 3A6 immunoprecipitated a biotinylated cell surface protein of 112 kDa which was detected by HRP-streptavidin. The same band of protein, either immunoprecipitated by 2D4 or immunoprecipitated by 3A6, was also recognized by anti-ALCAM monoclonal antibody 2D4 as well as sc-8548 and sc-8549 in western blot. The results confirmed all those antibodies bound to a common antigen, ALCAM, which was present in prostate cancer cell line, LNCAP.

### Example 2

Immunohistochemical Staining of ALCAM in Tissue Sections with Biotinylated Primary Anti-ALCAM Antibody

Cryosections were cut at 10 micron with Leica CM3050 and thaw-mounted onto slides. The sections were allowed to air-dry for at least for 30 minutes at room temperature. The sections were fix by immersing the slides in ethanol (pre-chilled at −20 degrees C.) and followed by air-drying overnight, or fixed with 4% paraformaldehyde for 5 minutes. The fixed slides can be used immediately or stored at −80 degrees C. freezer until use. To inactivate endogenous peroxidase activity, the slides were incubated in 1% hydrogen peroxide in methanol for 30 minutes at room temperature. Before primary antibody incubation, the slides were blocked for non-specific binding sites by incubation with 5% goat serum in PBS with 0.1% Tween 20 at room temperature for 60 minutes.

Biotinylated anti-ALCAM antibody was prepared as follows: purified monoclonal antibody was dialyzed against NaHCO₃ buffer, pH 9.0 overnight. 50 microl biotin labeling reagent (N-hydroxysuccininmidobiotin 2 mg/ml in DMSO, Pierce Cat# 20217) was added and mixed. The reaction mixture was allowed to react at room temperature on a rocking platform with gentle rocking overnight and then dialyzed against PBS to remove excess free biotin. The mixture was diluted in PBS containing 5% goat serum and 0.1% Tween 20 to a final concentration of approximately 1 microg/ml and added to the slide with an amount sufficient to cover the whole tissue sections, usually 0.5 ml per slide. The slides were sealed in a humid chamber saturated with PBS and incubated at 4 degrees C. overnight. At the end of incubation, the slides were washed by three rinses with PBS, five minutes each, to remove excess primary antibody. Then, the slides were incubated in Streptavidin-HRP solution (Sigma, Cat # S-5512, 10 microg/ml in PBS containing 5% goat serum and 0.1% Tween 20) for 1 hour at room temperature. Again, the slides were washed by three rinses with PBS, five minutes each, to remove excess streptavidin-HRP. Finally, the slides were washed with 2 rinses with peroxidase substrate buffer, sodium acetate buffer at pH 5.00 and developed in peroxidase substrate DAB/H₂O₂ at room temperature until the appropriate contrast was achieved, normally in a few minutes. The reaction was stopped by rinses in water to remove unreacted substrate. The slides were counterstained with hematoxylin and a coverslip was placed on the slide before microscopic examination. The staining results were examined and photographed under a Nikon microscope (Model E800). The results were scored as ‘+’ for weak positive staining, ‘2+’ for moderate positive staining, ‘3+’ for strong positive staining and ‘−’ for negative staining in Table 1 and 2. Table 1 shows anti-ALCAM antibody binding to various ovarian and prostate cancer tissues. Tissue samples #1 to #21 are ovarian cancer cell tissues and tissue samples #22 to #27 are prostate cancer tissue samples. Table 2 shows anti-ALCAM antibody binding to various normal human tissues.

TABLE 1
Binding of anti-ALCAM antibody to
ovarian cancers and prostate cancers

| Tissue | | Differentiation | |
|---|---|---|---|
| # | Cancer Type | Level | Result |
| 1 | Ovarian Serous Cancer | | — |
| 2 | Ovarian Serous Cancer | intermediate | 1-2+ |
| 3 | Ovarian Serous Cancer | intermediate | 2+ |
| 4 | Ovarian Serous Cancer | intermediate | 3+ |
| 5 | Ovarian Serous Cancer | intermediate | 2+ |
| 6 | Ovarian Serous Cancer | low | 1+ |
| 7 | Ovarian Serous Cancer | high | 3+ |
| 8 | Ovarian Serous Cancer | | — |
| 9 | Ovarian Serous Cancer | low | 3+ |
| 10 | Ovarian Serous Cancer | low | 3+ |
| 11 | Ovarian Serous Cancer | — | 2+ |
| 12 | Ovarian Serous Cancer | low | — |
| 13 | Ovarian Metastatic Cancer | | — |
| 14 | Ovarian Metastatic Cancer | | 3+ |
| 15 | Ovarian Metastatic Cancer | | 2+ |
| 16 | Ovarian Metastatic Cancer | | 2+ |
| 17 | Ovarian Mucinous Cancer | low | 1-2+ |
| 18 | Endometrioid Cancer | low | 2+ |
| 19 | Endometrioid Cancer | low | — |
| 20 | Malignant Brenner's | intermediate | — |
| 21 | Teratoma | | — |
| 22 | Prostate Cancer | | 3+ |
| 23 | Prostate Cancer | | 3+ |
| 24 | Prostate Cancer | | 3+ |
| 25 | Prostate Cancer | | 3+ |
| 26 | Prostate Cancer | | 2+ |
| 27 | Prostate Cancer | | 3+ |

TABLE 2
Distribution of ALCAM in normal human tissues

| Tissue Type | Results |
|---|---|
| Adrenal | Negative |
| Breast | Negative except for 1-2+ on glandular epithelium |
| C. cortex | Diffuse 1+ |
| Colon | Negative except for 2+ on nerves |
| Duodenum | Negative except for 2+ on nerve, 1-2+ on |
| | Brunner's gland and 1-2+ smooth muscle |
| Fallopian Tube | Negative except for 2+ on tubule epithelium |
| Heart | Negative except for rare 1+ in small vessels |
| Kidney | Negative |
| Liver | Negative except for 2+ on ductal epithelium |
| Lung | Negative except for 3+ on bronchial epithelium |
| Ovary | Negative except for focal 1+ cystic epithelial |
| | and stroma |
| Pancreas | Negative except for 2+ on nerves, 1+ acinar |
| Spleen | Negative |
| Skeletal Muscle | Negative |
| Skin | Negative except for 2+ sweat gland |
| Stomach | Negative except for 1+ glandular epithelium |
| Uterus | Negative except for 3+ glandular epithelium |

### Example 3

Binding of Anti-ALCAM to Pancreatic Cancers

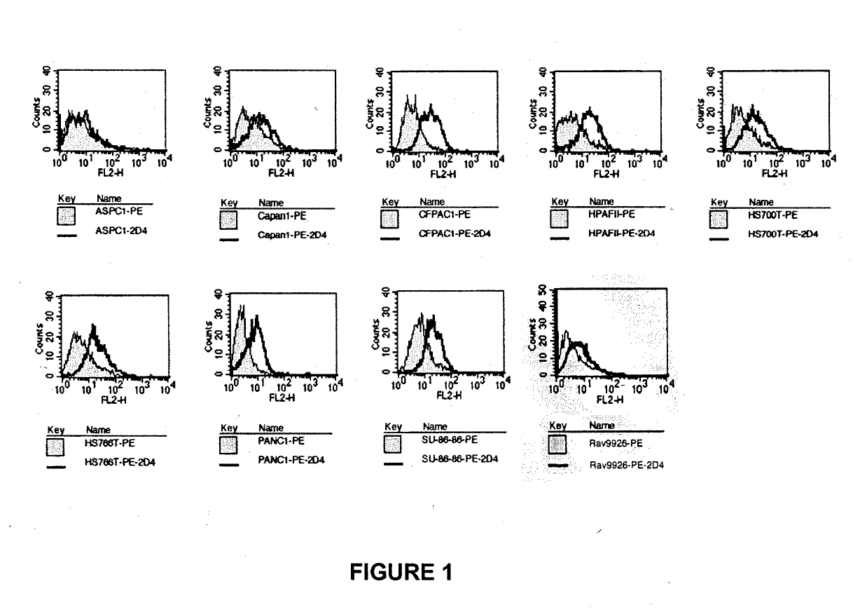

The binding of the anti-ALCAM monoclonal antibody 2D4 to pancreatic cancer cell lines (AsPC-1 (ATCC #CRL-1682), Capan-1 (ATCC #HTB-79), CFPAC-1 (ATCC #CRL-1918), HPAF-II (ATCC #CRL-1997), HS700T (ATCC #HTB-147), HS766T (ATCC #HTB-134), PANC1 (ATCC #CRL-1469), SU.86.86 (ATCC #CRL 1837 ), and Rav9926 (proprietary pancreatic cancer cell line) was analyzed by a fluorescence activated cell sorter in live cells. In each histogram, shown in FIG. 1, the gray, filled-in curve represents non-specific binding of fluorescence labeled anti-human IgG Fc to each cell line without primary antibody. The black curve shows FACS sorting of cells bound to anti-ALCAM antibody. A shift of the black curve from the gray curve to the right indicated specific binding of the anti-ALCAM antibody to the respective cell line and therefore implicated that the cell line express ALCAM on cell surface. Eight of the nine pancreatic cell lines tested express ALCAM (Capan-1, CFPAC-1, HPAF-II, HS700T, HS766T, PANC1, SU-86-86, and Rav9926.

### Example 4

Binding of Anti-ALCAM Lung Cancers

The binding of anti-ALCAM to lung cancer cell line SK-MES-1 (ATCC #HTB 58), a primary squamous cell carcinoma, was analyzed by live cell enzyme linked immunoassay. SK-MES-1 cell line was grown in F12/DMEM medium supplemented with 10% fetal bovine serum to confluence in tissue culture treated 96 well tissue culture plates (Falcon cat no. 354075). Cells were washed with tissue culture medium and then incubated with or without 10 microg/ml anti-ALCAM antibody (MAb 2D4) in Hank's balanced salt solution (HBSS) containing 1% BSA and 0.1% sodium azide for 1 hour at room temperature. The cells were then washed three times with 100 microl per well of HBSS before incubation with 50 microl per well horse radish peroxidase (HRP) conjugated donkey anti-human IgG heavy and light chain specific antibodies at a concentration of 0.8 microg/ml diluted in HBSS for 30 minutes room temperature. The cells were finally washed three times with HBSS and incubated in 100 microl TMB substrate (KPL Cat No. 50-65-00 and 50-76-01) for 5 minutes and stopped by addition of 100 microl per well of 1 M phosphoric acid. The developed plates were read at O.D. 450 nm. The result showed that MAb 2D4 generated an O.D.450 value of 0.356 with a standard error value of 0.064, when control (without MAb 2D4) O.D.450 was set as blank, indicating SK-MES-1 cells expressed ALCAM on the cell surface.

### Example 5

Anti-ALCAM Antibody Enhanced Vascularization in Graft of ALCAM Positive Cells in Nude Mice

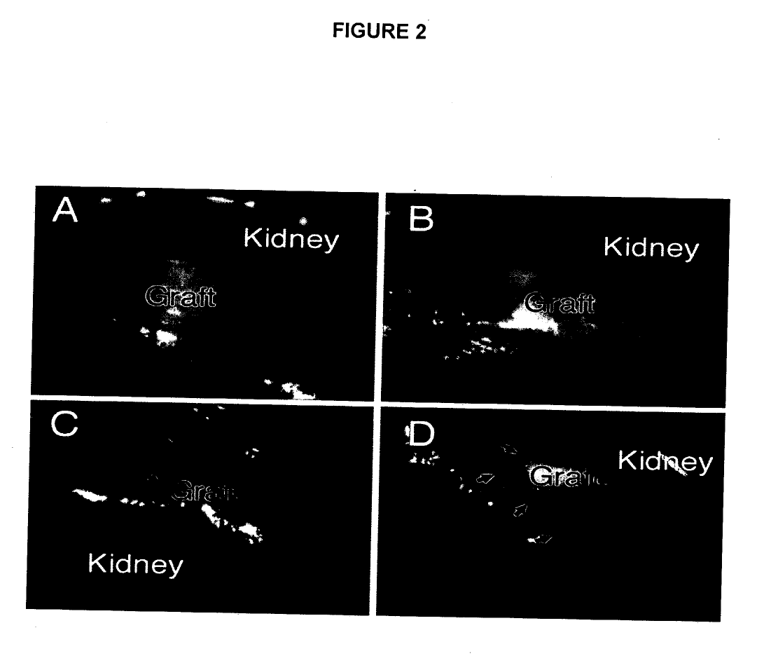

Rav 9926 cells (proprietary human pancreatic tumor cell line) were grafted under the kidney capsule in nude (nu/nu) mice. The graft was allowed to grow for one week. Anti-ALCAM monoclonal antibody, clone 2D4, was injected intraperitoneally at 100 mg/kg dose every two days for three injections. Control mice were injected with an excipient. At day 10, the animals were euthanized and the kidneys with grafts were examined. FIG. 2 shows that the cell grafts in control mice were white and translucent while the cell grafts in mice injected with 2D4 monoclonal antibody were entangled with dense vasculature, as indicated by the arrows. The results indicated that 2D4 monoclonal antibody enhanced angiogenesis in cells of tissue grafts.

Since ALCAM may be present on the surface of endothelial cells, the effect of ALCAM antibody addition to endothelial cells was evaluated. Human umbilical vein (Cat. # CC2519), adult aortic (Cat. # CC2535) and neonatal dermal (Cat. # CC-2516) microvascular endothelial cells were obtained from the Clonetics division of BioWhittaker and tested at an early passage. Cells were plated at a density of approximately 1500 cells per well in 96 well plate and were incubated with or without 50 microg/ml of 2D4 for 4 days, and growth was measured by staining with crystal violet and reading optical density. The presence of the ALCAM antibody had no effect on the growth of any of these endothelial cells. It can therefore be concluded that the increased vascularization seen in vivo after treatment with ALCAM antibodies was most likely not due to a direct effect on endothelial cell growth. Additional experiments have shown that the 2D4 MAb exhibits species specificity and does not bind to mouse ALCAM. In certain embodiments of this invention, and without being limited to a particular mechanism of action, the neovascularization observed in vivo resulted from the ALCAM antibody-dependent release of vascularization-promoting substance by the tumor cells, which secondarily cause neovascularization.

### Example 6

Immunohistochemical Staining of ALCAM in Tissue Sections with Biotinylated Anti-ALCAM Antibody mKID2

Tissue sections were prepared and stained with an anti-ALCAM antibody mKID2 using methods described in Example 2. The anti-ALCAM antibody mKID2 was produced from the hybridoma deposited in the American Type Culture Collection (ATCC) 10801 University Blvd., Manassas Va. 20110-2209 on Jun. 21, 2002 with a Patent Deposit Designation of PTA-4478. The staining results were examined and photographed under a Nikon microscope (Model E800). The results were scored as ‘+’ for weak positive staining, ‘2+’ for moderate positive staining, ‘3+’ for strong positive staining and ‘−’ for negative staining in Table 1 and 2. Table 3 shows anti-ALCAM antibody mKID2 binding to various colon, lung, prostate, and breast cancer tissues.

TABLE 3
Distribution of ALCAM in tumors (mKID2 antibody)

| Tissue # | Tumor type | IHC result |
|---|---|---|
| 29 | Colon Adenocarcinoma | Focal 1-2+ |
| 30 | Colon Adenocarcinoma | Variable 1-3+ |
| 31 | Colon Adenocarcinoma | 1+ |
| 32 | Colon Adenocarcinoma | +/− |
| 33 | Colon Adenocarcinoma | −− |
| 34 | Lung, adenocarcinoma | 1+ |
| 35 | Lung, squamous cell carcinoma | Focal 1+ |
| 36 | Lung, non-small cell carcinoma | 2-3+ |
| 37 | Lung, non-small cell carcinoma | 1-2+ |
| 38 | Lung, non-small cell carcinoma | Focal 1+ |
| 39 | Lung, non-small cell carcinoma | +/− |
| 40 | Prostate adenocarcinoma | 2+ |
| 41 | Prostate adenocarcinoma | Focal 1+ |
| 42 | Prostate adenocarcinoma | 1+ |
| 43 | Prostate adenocarcinoma | +/− |
| 44 | Breast, adenocarcinoma | 1-2+ |
| 45 | Breast, adenocarcinoma | +/− |

+/− staining equivocal

### Example 7

Internalization of an Anti-ALCAM Antibody and Toxin-conjugated Anti-mouse IgG

MAb-ZAP (Advanced Targeting Systems, San Diego, Calif.) is anti-mouse IgG conjugated to saporin, a toxin that inhibits protein synthesis. This toxin is impermeable to the cell membrane. If a monoclonal antibody is bound to a cell-surface antigen that is internalizable, the toxin-conjugate can bind to the bound monoclonal and be internalized, eventually killing the cell. Being dependent upon internalization for demonstration of toxic activity, the MAb-ZAP can serve to evaluate whether or not a given surface antigen will serve as a suitable target for any toxin that is dependent upon internalization to express cell toxic effects. As such, the MAb-ZAP serves as a model for such internalization-dependent toxins such as maytansinoids and calicheamicins.

For testing the internalization of an anti-ALCAM antibody and saporin conjugated anti-mouse IgG and the effect of inhibiting tumor cell growth after internalization of saporin, monoclonal anti-ALCAM antibody mKID2 was used in the assay. Human ovarian cancer cells, SKOV3 (ATCC #HTB 77) were removed from stock flasks with 10 mM EDTA and centrifuged. Cells were resuspended at 50,000/ml in appropriate medium and 100 microl plated per well in 96 well plates. Antibody mKID2 was added immediately to appropriate wells as a 10× concentrate, to make a final concentration of 10 microg/ml. After 15 minutes at room temperature MAb-ZAP (Cat. # IT-04, Advanced Targeting Systems, San Diego Calif.) was added to appropriate wells as 10× concentrate, to make final concentrations from 0.001 pM to 10⁴ pM. After 4 days growth, MTT was added (stock 5 mg/ml PBS, 1:10 dilution in well) for 4 hrs at 37° C. The medium was then removed from all wells and 100 microl/well DMSO was added. The plates were gently swirled to solubilize the blue MTT precipitate and the plates were read in a plate reader at 540 nm.

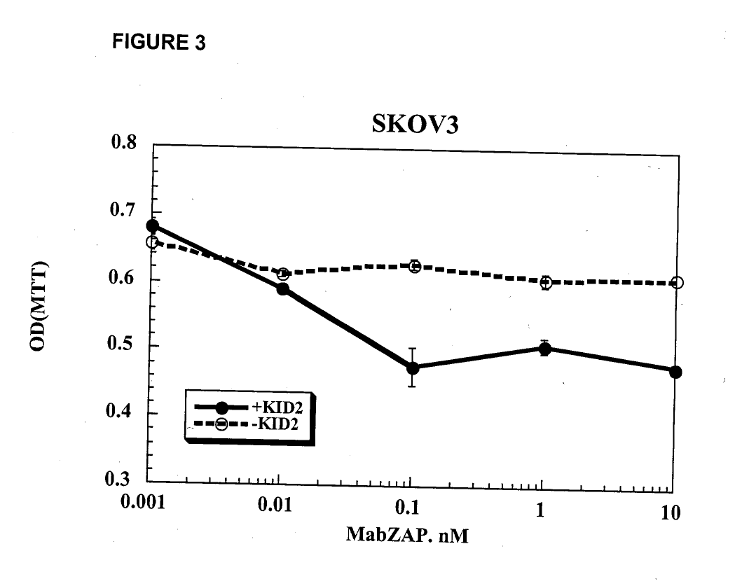

As shown in FIG. 3, there was a decrease in MTT staining in SKOV3 cells in the presence of mKID2 as compared to the staining in the absence of mKID2 when MAB-ZAP was added above 100 pM, indicating the growth of human ovarian cells SKOV3 was inhibited in the presence of mKID2 and MAb-ZAP, and mKID2 and toxin-conjugated anti-mouse IgG were internalized in SKOV3.

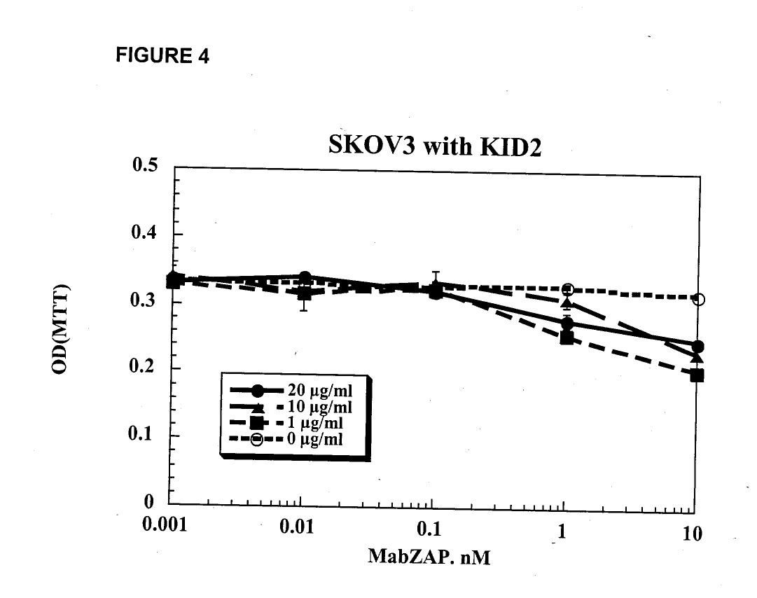

Similar tests were performed in human ovarian cancer cells SKOV3 with different concentration of mKID2. As shown in FIG. 4, there were decreases in MTT staining in SKOV3 cells in the presence of 1 microg/ml, 10 microg/ml, and 20 microg/ml mKID2 combined with 10 nM Mab-ZAP.

It is understood that the examples and embodiments described herein are for illustrative purposes only and that various modifications or changes in light thereof will be suggested to persons skilled in the art and are to be included within the spirit and purview of this specification. All publications, patents and patent applications cited herein are hereby incorporated by reference in their entirety for all purposes to the same extent as if each individual publication, patent or patent application were specifically and individually indicated to be so incorporated by reference.

We claim:

1. A substantially purified immunoglobulin polypeptide or an antigen binding fragment thereof, that specifically binds to activated leukocyte cell adhesion molecule (ALCAM), and has at least one or more of the following characteristics:
a. the ability to bind to ALCAM on a cancer cell;
b. ability to bind to a portion of ALCAM that is exposed on the surface of a living cell in vitro or in vivo;
c. ability to deliver a therapeutic agent or detectable marker to a cancer cell expressing ALCAM;
d. ability to deliver a therapeutic agent or detectable marker into a cancer cell expressing ALCAM;
e. ability to enhance blood vessel generation in an individual; and
f ability to reduce blood vessel generation in an individual.

2. The purified immunoglobulin polypeptide or antigen binding fragment of claim 1, wherein said cancer cell is selected from the group consisting of ovarian, prostate, pancreatic, lung, colon, or breast cancer cells.

3. An isolated nucleic acid sequence coding for the immunoglobulin polypeptide or antigen binding fragment thereof of claim 1.

4. The nucleic acid of claim 3, wherein the nucleic acid is operably linked to a promoter.

5. The nucleic acid of claim 4, wherein the promoter and the nucleic acid are contained in an expression vector.

6. The nucleic acid of claim 3, wherein the polypeptide is a monoclonal antibody.

7. A cell line transfected, transformed, or infected with a vector containing a nucleic acid of claim 3.

8. A method of producing a substantially purified immunoglobulin polypeptide, or an antigen binding fragment thereof, comprising the steps of:
a. Growing a cell line transformed with the nucleic acid of claim 3 under conditions in which the immunoglobulin polypeptide or antigen binding fragment is expressed; and
b. Harvesting the expressed immunoglobulin polypeptide or fragment.

9. The method of claim 8, wherein the cell line is a hybridoma.

10. The method of claim 9, wherein the hybridoma is ATCC No. PTA-4478.

11. The method of claim 8, wherein the immunoglobulin polypeptide is a monoclonal antibody.

12. A pharmaceutical composition comprising a therapeutically effective dose of the purified immunoglobulin or antigen binding fragment of any of claims 1 or 15-16, together with a pharmaceutically acceptable carrier.

13. A pharmaceutical composition comprising a therapeutically effective dose of a monoclonal antibody or an antigen binding fragment thereof that specifically binds to ALCAM, and has at least one or more of the following characteristics:
a. the ability to bind to ALCAM on a cancer cell;
b. ability to bind to a portion of ALCAM that is exposed on the surface of a living cell in vitro or in vivo;
c. ability to deliver a therapeutic agent or detectable marker to a cancer cell expressing ALCAM;
d. ability to deliver a therapeutic agent or detectable marker into a cancer cell expressing ALCAM;
e. ability to enhance blood vessel generation in an individual; and
f ability to reduce blood vessel generation in an individual, together with a pharmaceutically acceptable carrier.

14. The pharmaceutical composition of claim 13, wherein the composition comprises an additional therapeutic moiety.

15. A purified immunoglobulin polypeptide or an antigen binding fragment thereof that specifically binds to ALCAM and reduces neovascularization.

16. A purified immunoglobulin polypeptide or an antigen binding fragment thereof that specifically binds to ALCAM and enhances neovascularization.

17. An isolated cell line designated as ATCC No. PTA-4478 or progeny thereof.

18. A method for delivering a chemotherapeutic agent to a cancer cell comprising administering a composition comprising an anti-ALCAM antibody associated with the chemotherapeutic agent, wherein the cancer cell is selected from the group consisting of ovarian, prostate, pancreatic, lung, colon, and breast cancer cells.

19. The method of claim 18, wherein the chemotherapeutic agent is administered to an individual.

20. The method of claim 18, wherein the anti-ALCAM antibody is an antibody expressed by cell line ATCC No. PTA-4478 or progeny thereof.

21. A method of inhibiting growth of cancer cells in an individual comprising administering to the individual an effective amount of a composition comprising an anti-ALCAM antibody associated with a chemotherapeutic agent to the individual, wherein the cancer cells are selected from the group consisting of ovarian, prostate, pancreatic, lung, colon, and breast cancer cells.

22. The method of claim 21, wherein the chemotherapeutic agent is delivered into the cancer cells.

23. The method of claim 21 wherein the anti-ALCAM antibody is a monoclonal antibody expressed by cell line ATCC No. PTA-4478 or progeny thereof.

24. A method for detecting the presence or absence of a cancer cell in an individual comprising contacting cells from the individual with an anti-ALCAM antibody, and detecting a complex of ALCAM from the cells and the antibody, if any, wherein the cancer cell is selected from the group consisting of ovarian, prostate, pancreatic, lung, colon, and breast cancer cells.

25. An agent that blocks at least one of the following interactions between ALCAM and an ALCAM binding partner:
a. the ability to bind to ALCAM on a cancer cell;
b. ability to bind to a portion of ALCAM that is exposed on the surface of a living cell in vitro or in vivo;
c. ability to deliver a therapeutic agent or detectable marker to a cancer cell expressing ALCAM;
d. ability to deliver a therapeutic agent or detectable marker into a cancer cell expressing ALCAM;
e. ability to enhance blood vessel generation in an individual; and
f. ability to reduce blood vessel generation in an individual.

26. A pharmaceutical composition comprising a therapeutically effective dose of an agent according to claim 25, together with a pharmaceutically acceptable carrier.
